(19) United States
(12) Patent Application Publication
(10) Pub. No.: US 2011/0110892 A1
(43) Pub. Date: May 12, 2011

(54) VECTORS FOR DELIVERING DISEASE NEUTRALIZING AGENTS

(75) Inventor: Ronald C. Desrosiers, Southborough, MA (US)

(73) Assignee: President and Fellows of Harvard College, Cambridge, MA (US)

(21) Appl. No.: 12/934,505

(22) PCT Filed: Mar. 24, 2009
(86) PCT No.: PCT/US09/01873

§ 371 Date: Dec. 13, 2010

Related U.S. Application Data
(62) Division of application No. 61/039,099, filed on Mar. 24, 2008.

(51) Int. Cl. A61K 35/76; C12N 7/01; A61P 31/00

(52) U.S. Cl. 424/93.2; 435/235.1

(57) ABSTRACT

The invention relates in some aspects to recombinant herpes viruses and their use for expressing and delivering one or more disease-neutralizing agents to a subject to prevent or treat a disease in the subject. Some aspects of the invention relate to virus compositions and formulations comprising recombinant herpes viruses (e.g., recombinant gamma herpes viruses) that expresses one or more disease-neutralizing agents such as antibodies or other agents that can interfere with disease infection or progression. In some aspects, the invention relates to methods for preventing or treating an immunodeficiency virus associated disease. In some aspects, the invention relates to methods for preventing or treating AIDS.

### RELATED APPLICATIONS

This application claims the benefit under 35 U.S.C. §119(e) of U.S. provisional patent applications Ser. No. 61/039,099, filed on Mar. 24, 2008, the contents of which are incorporated herein by reference in their entirety.

### FIELD OF THE INVENTION

The invention relates to methods for protecting subjects from infection by one or more disease-associated organisms.

### BACKGROUND OF INVENTION

More than twenty years have passed since the discovery of HIV and its association with AIDS. Still, despite much scientific and clinical investigation there is no effective vaccine to protect vulnerable populations around the globe and perhaps treat those living with the disease. Development of an effective vaccine against HIV/AIDS faces severe scientific obstacles and vaccine approaches that are currently in human testing have provided little or no protection in monkeys against challenge by simian immunodeficiency virus (SIV).

### SUMMARY OF INVENTION

Aspects of the invention provide an alternative approach to traditional immunization. Aspects of the invention relate to methods and compositions for delivering disease neutralizing agents to subjects in order to protect them from infection or disease progression. The neutralizing agents may interfere with infection and/or disease progression by interacting directly with a disease-associated organism (e.g., a virus or microbial organism), by interacting with one or more host factors associated with infection or disease progression, or a combination thereof. In some aspects, the invention provides recombinant herpes viruses (e.g., gamma herpes viruses) that express one or more disease neutralizing agents. Some aspects of the invention relate to prophylactic compositions and formulations comprising recombinant herpes viruses that expresses immunodeficiency virus neutralizing agents. In some aspects, the invention relates to methods for preventing or treating an immunodeficiency virus associated disease. In some aspects, the invention relates to methods for preventing or treating AIDS. In some embodiments, recombinant HI-IV-8 is used as a vaccine to protect humans against HIV/AIDS, and/or against other infectious diseases.

Aspects of the invention are based, at least in part, on the discovery that recombinant herpes viruses (e.g., gamma herpes viruses) represent a promising new vector for delivering prophylactic or therapeutic agents to a subject in an amount sufficient to neutralize one or more infectious organisms and prevent them from infecting a subject and/or proliferating within the subject. Aspects of the invention may be used, for example, to prevent and/or treat a viral infection such as HIV (and thereby protect against immunodeficiency virus associated diseases, such as AIDS). It should be appreciated that the term “treat” as used herein can refer to managing a disease by preventing or slowing disease progression.

Aspects of the invention relate to providing vectors (e.g., herpes vectors) that can be used for persistent delivery of one or more neutralizing agents to a subject. Persistence within the body of a subject can be useful for delivering prophylactic or therapeutic molecules to a subject for extended periods of time. Accordingly, aspects of the invention can be used to provide long term protection to a subject (e.g., a healthy subject who is at risk of infection). Other aspects of the invention can be used to provide long term persistent treatment to a subject (e.g., a diseased subject who has an infection susceptible to treatment with the neutralizing agents).

As used herein, to “interfere” may mean to inhibit (e.g., to reduce). It should be appreciated that, in some embodiments, an inhibition may be a reduction of one or more properties (e.g., proliferation of a pathogenic virus) or symptoms (e.g., decreased immune function) of infection in an individual relative to a control individual (e.g., a non-treated individual, e.g., an individual that has not been treated with a neutralizing agent). An inhibition may be, for example, at least a 5%, 10%, 20%, 30%, 40%, 50%, 60%, 70%, 80%, 90%, or higher or lower percentage reduction.

In some embodiments, neutralizing agents may be broadly neutralizing antibodies (e.g., broadly neutralizing monoclonal antibodies). Neutralizing antibodies may include antibodies or antigen-binding fragments (e.g., F(ab′)₂, Fab, Fd, or Fv fragments). In some embodiments, the neutralizing antibody is a monoclonal antibody, such as a chimeric, human, or humanized antibody, or a single chain antibody. Exemplary neutralizing antibodies are discussed in Karlsson Hedestam G B, et al., Nat Rev Microbiol. 2008 February; 6(2):143-55; Pantophlet R, Burton D R. Annu Rev Immunol. 2006; 24:739-69; Burton D R, et al., Proc Natl Acad Sci USA. 2005 Oct. 18; 102(42):14943-8; and Burton D R. Nat Rev Immunol. 2002 September; 2(9):706-13. Examples of anti-SIV rhesus monoclonal antibodies include SIV239, SIVE543, and SIV251. Further examples are provided in Johnson W. E., et al., Journal of Virology, September 2003, p. 9993-10003; Vol. 77, No. 18 (2003) (disclosing rhesus macaque monoclonal antibodies recognizing SIV envelope glycoproteins, e.g., at page 9994 column 2, paragraph 5). In some embodiments, neutralizing agents are antibodies, or antigen-binding antibody fragments, that specifically bind to an amino acid sequence set forth in GenBank Accession# NP_{—}057856.1 (Envelope surface glycoprotein gp160, precursor protein) or NP_{—}579894.2 (Envelope surface glycoprotein gp120). It should be appreciated that neutralizing agents are not limited to antibodies, but could be other molecules that interfere with infection, viral proliferation, or disease progression.

Neutralizing agents also may be neutralizing nucleic acids (e.g., aptamers, ribozymes, siRNA, or other functional nucleic acids) or polypeptides that can interfere with infection or disease progression. For example, molecules (e.g., antibodies or small peptides) that inhibit CCR5 and/or CD4 may be used to inhibit viral entry to protect against HIV infections and/or proliferation. Peptide or antibody based HIV entry inhibitors that are in development and in clinical trials also may be used. For example, gp41 targeting peptides such as Enfuvirtide (Fuzeon, T20) and Sifuvirtide; CCR5 targeting agents such as the monoclonal antibody, PRO 140; CD4 targeting agents such as the monoclonal antibody, TNX-355; and gp120 targeting agents, such as PRO 542 (CD4-IgG2) and the b12 antibody may be encoded in a recombinant viral vector of the invention and used to establish a persistent protection against HIV. Other peptide based inhibitors of HIV entry are exemplified in Peter S. Kim et al., Peptide inhibitors of HIV entry—U.S. Pat. No. 6,747,126; Welch B D et al., Potent D-peptide inhibitors of HIV-1 entry, PNAS, Oct. 23, 2007, vol. 104, no. 43, 16828-16833; J. Esté, A. Telenti HIV entry inhibitors; The Lancet, Volume 370, Issue 9581, Pages 81-88; Root M J, et al., Protein Design of an HIV-1 Entry Inhibitor Science 2 Feb. 2001: Vol. 291. no. 5505, pp. 884-888; G. B. Melikyan et al., Membrane-Anchored Inhibitory Peptides Capture Human Immunodeficiency Virus Type 1 gp41 Conformations That Engage the Target Membrane prior to Fusion. (2006) J. Virol. 80, 3249-3258; E. de Rosny, et al., (2004)Binding of the 2F5 Monoclonal Antibody to Native and Fusion-Intermediate Forms of Human Immunodeficiency Virus Type 1 gp41: Implications for Fusion-Inducing Conformational Changes. J. Virol. 78, 2627-2631; J. G. Joyce, et al., Enhancement of alpha-Helicity in the HIV-1 Inhibitory Peptide DP178 Leads to an Increased Affinity for Human Monoclonal Antibody 2F5 but Does Not Elicit Neutralizing Responses in Vitro. IMPLICATIONS FOR VACCINE DESIGN. (2002) J. Biol. Chem. 277, 45811-45820; J. M. Louis, et al., Design and Properties of NCCG-gp41, a Chimeric gp41 Molecule with Nanomolar HIV Fusion Inhibitory Activity. (2001) J. Biol. Chem. 276, 29485-29489; Tamamura H, et al., Development of anti-HIV agents targeting dynamic supramolecular mechanism: entry and fusion inhibitors based on CXCR4/CCR5 antagonists and gp41-C34-remodeling peptides. Curr HIV Res. 2005 October; 3(4):289-301; Ruff M R, et al., Update on D-ala-peptide T-amide (DAPTA): a viral entry inhibitor that blocks CCR5 chemokine receptors. Curr HIV Res. 2003 January; 1(1):51-67. Review and Johnson, W. E., et al., Journal of Virology, September 2003, p. 9993-10003, Vol. 77, No. 18.

In some embodiments, neutralizing agents are nucleic acids (e.g., siRNA, shRNA, miRNA) that specifically hybridize to a sequence set forth in Genbank Accession #NC_{—}001802, which corresponds to the complete genome of human immunodeficiency virus 1, and inhibit the expression of a gene (e.g., a gene in Table 3) associated with that sequence. For example, a neutralizing agent may specifically hybridize to a sequence that encodes the HIV1 envelope protein (a sequence between positions 5771 and 8341 of NC_{—}001802) and inhibit expression of the envelope protein.

Similarly, other decoy molecules may be used to inhibit viral entry (e.g., to inhibit HIV viral entry) and protect a subject against infection or disease progression. It should be appreciated that in some embodiments compositions of the invention should persist in amounts sufficient to express effective levels of the one or more neutralizing agents. An advantage of broadly neutralizing antibodies or other broadly neutralizing agents is that they can be effective against a range of different types or variants of an infectious organism (e.g., HIV sequence variants). It should be appreciated that a neutralizing antibody or other binding agent that binds to an epitope on a pathogenic organism (e.g., a virus) may target the pathogenic organism for destruction by the immune system, removal in the glomeruli, or other processing event that removes the pathogenic organism from a subject and reduce the risk of infection. These and other aspects of the invention are described in more detail herein.

According to some aspects, the invention includes compositions comprising a recombinant herpes virus that encodes at least one neutralizing agent, wherein the neutralizing agent interferes with infection or proliferation of a pathogenic organism in a subject.

In some embodiments of the compositions disclosed herein, the pathogenic organism is a virus. In some embodiments, the virus is an immunodeficiency virus. In specific embodiments, the immunodeficiency virus is selected from HIV-1 and HIV-2. In certain embodiments, the HIV-1 is a subtype selected from: A, B, C, D, F, H, and O. In certain other embodiments, the HIV-2 is a subtype selected from: A and B.

In some embodiments of the compositions disclosed herein, the herpes virus is a gamma herpes virus. In specific embodiments, the gamma herpes virus is HHV-8.

In some embodiments of the compositions disclosed herein, the neutralizing agent is a nucleic acid or protein that interferes with infection of the subject by the pathogenic organism.

In some embodiments of the compositions disclosed herein, the neutralizing agent inhibits CCR5 and/or CD4.

In some embodiments of the compositions disclosed herein, the neutralizing agent is a decoy molecule that inhibits viral entry into a host cell in the subject.

In some embodiments of the compositions disclosed herein, the neutralizing agent is an antibody. In specific embodiments, the antibody is a broadly neutralizing antibody that binds to one or more HIV antigens.

In some embodiments of the compositions disclosed herein, the one or more HIV antigens are Gag, Pol, Env, Tat, Rev, Vif, Vpr, Vpu, Nef, or Vpx antigens.

In some embodiments of the compositions disclosed herein, the neutralizing agent is encoded by a nucleic acid that is operably joined to a promoter selected from: an SV40 early promoter, an SV40 late promoter, a CMV immediate early promoter, an EF1 promoter, and a promoter of an endogenous gamma herpesvirus gene.

In some embodiments of the compositions disclosed herein, the subject is human.

In some embodiments, the foregoing compositions are pharmaceutical compositions that comprise a pharmaceutically acceptable carrier.

In some aspects, the invention provides methods of protecting a subject from infection by a pathogenic organism. In some embodiments, the methods comprise administering to a subject at risk of infection by a pathogenic organism an effective amount of a recombinant herpes virus that encodes at least one neutralizing agent, wherein the neutralizing agent interferes with infection of the subject by the pathogenic organism.

In some aspects, the invention provides methods of reducing the viral load of an immunodeficiency virus in a subject. In some embodiments, the methods comprise administering to a subject infected with an immunodeficiency virus, an effective amount of a recombinant herpes virus that encodes at least one neutralizing agent, wherein the neutralizing agent interferes with proliferation of the immunodeficiency virus in the subject.

In some embodiments, the recombinant herpes virus used in the foregoing methods is provided in a pharmaceutical composition.

### BRIEF DESCRIPTION OF DRAWINGS

FIG. 1 depicts an alignment of HHV-8 and RRV genomes.

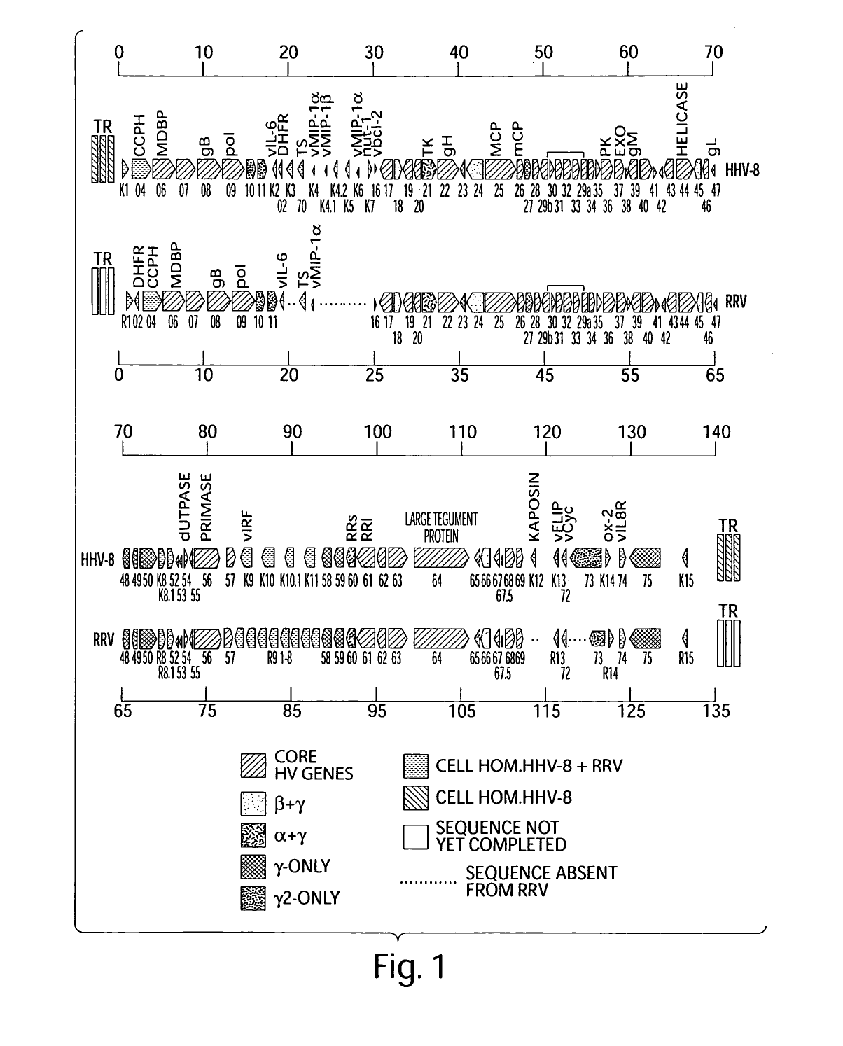

FIG. 2 depicts an exemplary site for transgene insertion in RRV. In this depiction, the transgene is inserted 183 by upstream from the R1 promoter.

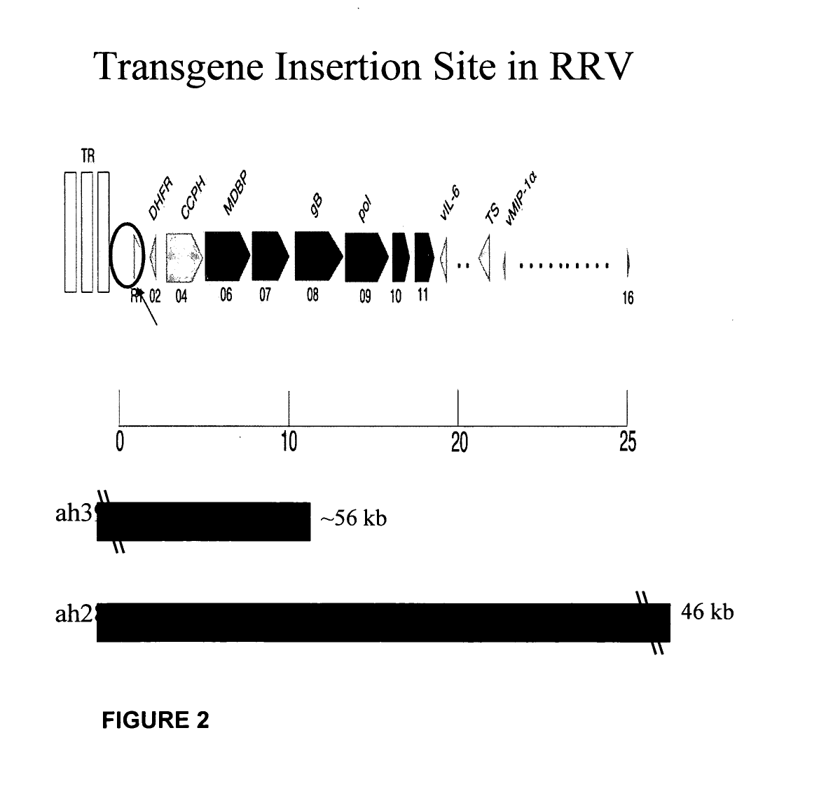

FIG. 3 depicts recombinant rhadinoviruses expressing immunodeficiency virus antigens.

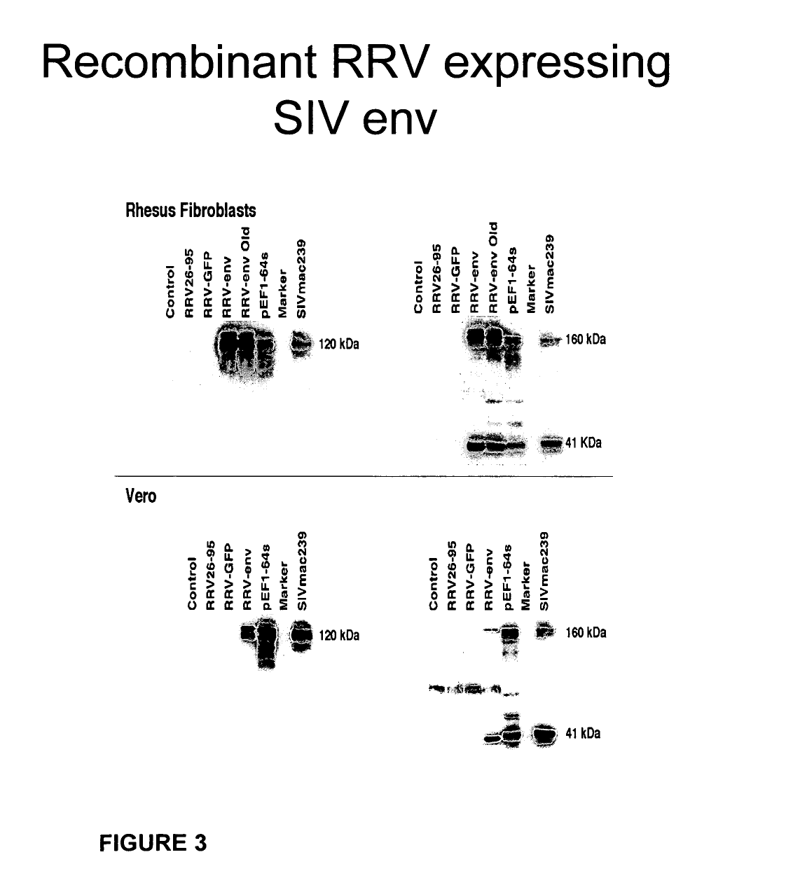

FIG. 4 depicts recombinant rhadinoviruses infection of target cells and expression of immunodeficiency virus antigens. Expression of RRV-env and RRV-env-Old are shown in rhesus fibroblasts and Vero cells.

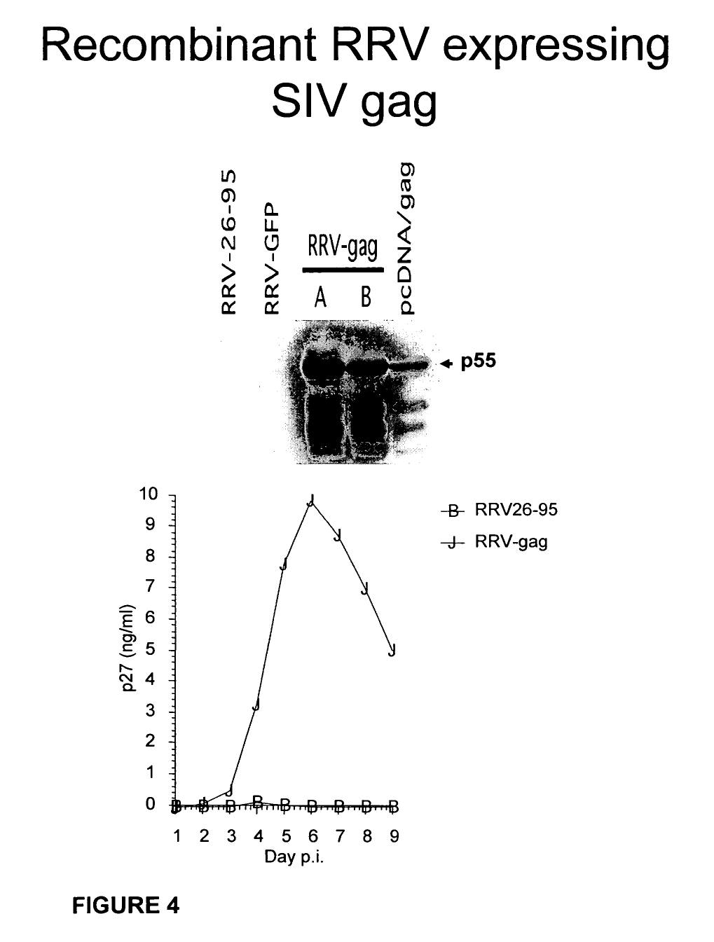

FIG. 5 depicts recombinant rhadinovirus infection of target cells and expression of immunodeficiency virus antigens aT various intervals (1, 2, 3, 4, 5, 6, 7, 8 and 9 days) after infection Expression of RRV-gag and RRV26-95 is shown.

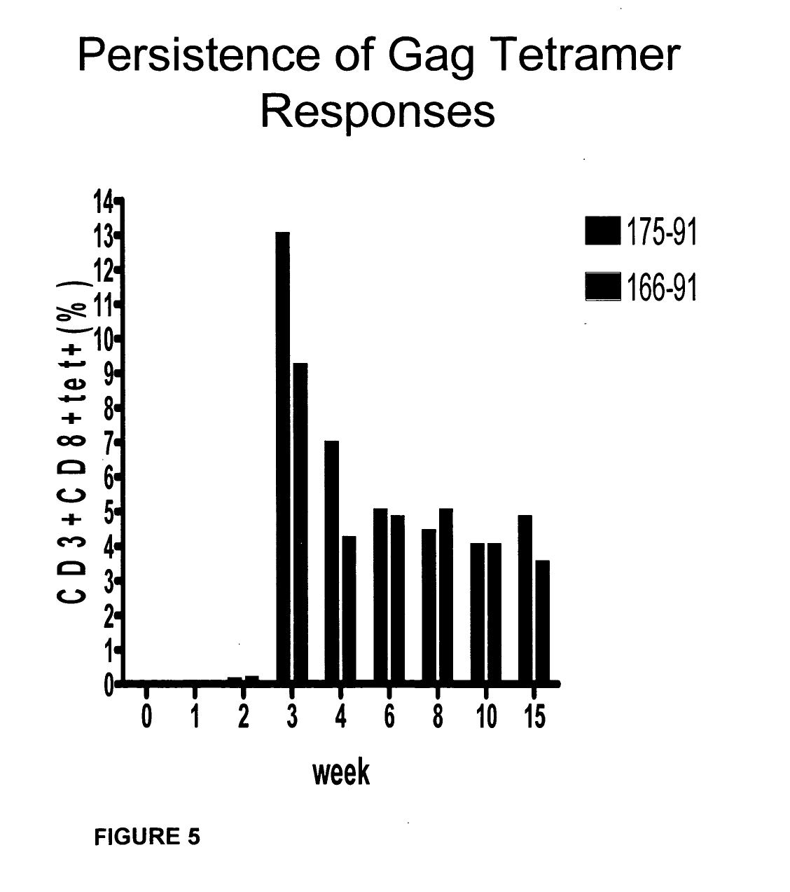

FIG. 6 depicts the persistence of tetramer responses to Gag in rhesus monkeys at various intervals (1, 2, 3, 4, 5, 6, 7, 8, 10, and 15 days) post treatment (inoculation) with RRV expression of Gag. Bars reflect the percentage of CD3+, CD8+, and Tetramer+ T-cells in independent experiments (175-91 and 166-91).

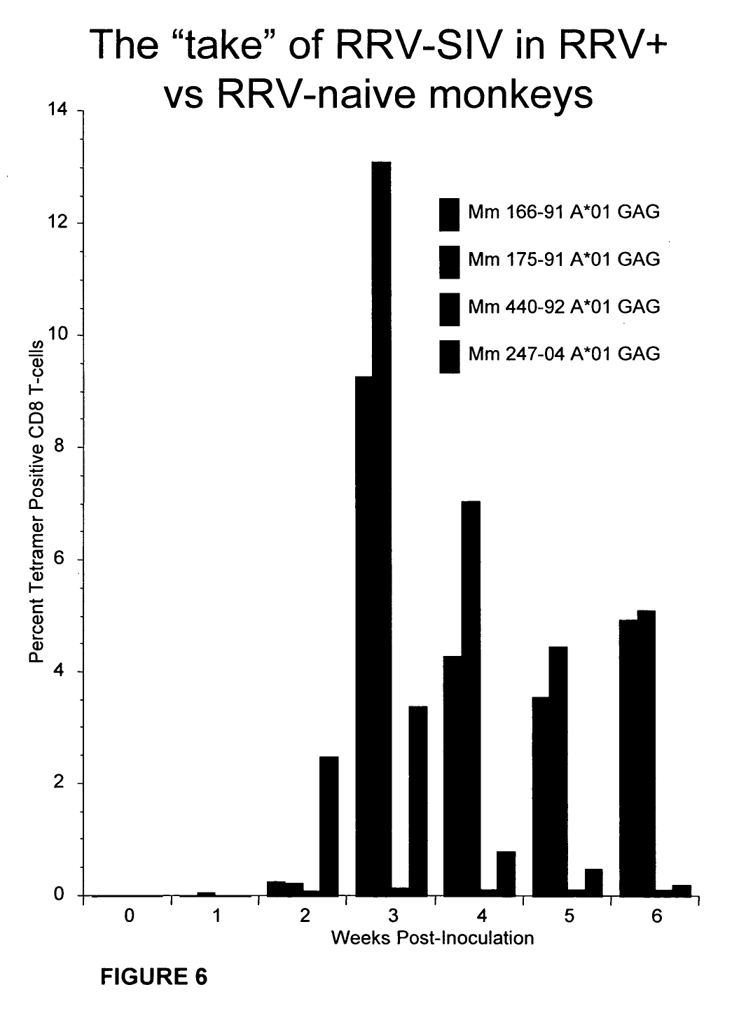

FIG. 7 depicts an IFN-Gamma ELISPOT assay in 5 five monkeys pre-inoculation and at 3 and 9 weeks post inoculation.

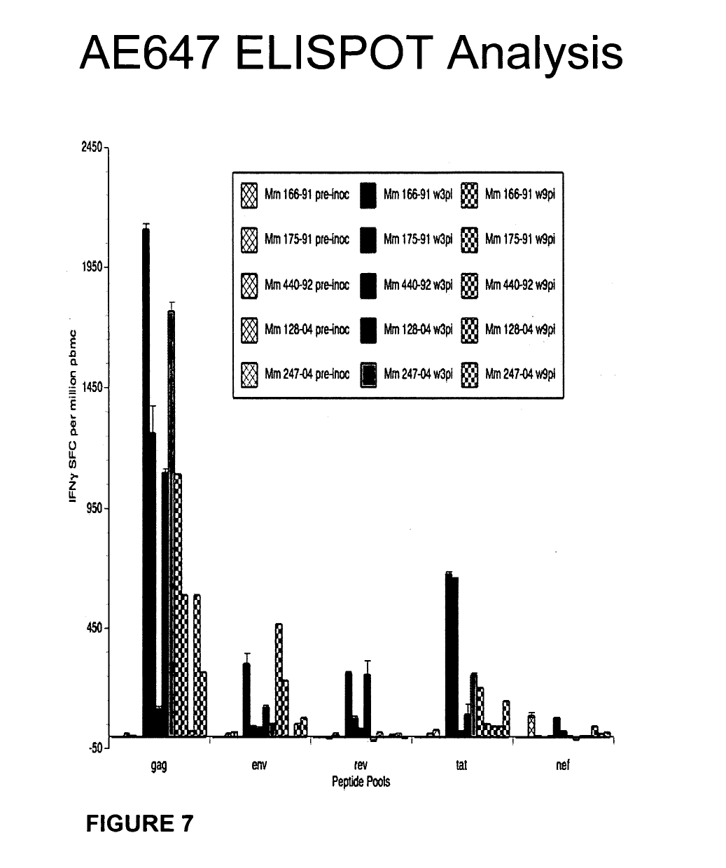

FIG. 8 depicts Gag-specific CD8+ T cells in RRV-vaccinated animals that display an effector memory phenotype (CD28-CCR7-Perforin+) five weeks after inoculation. Similar results are observed at 12 weeks post inoculation.

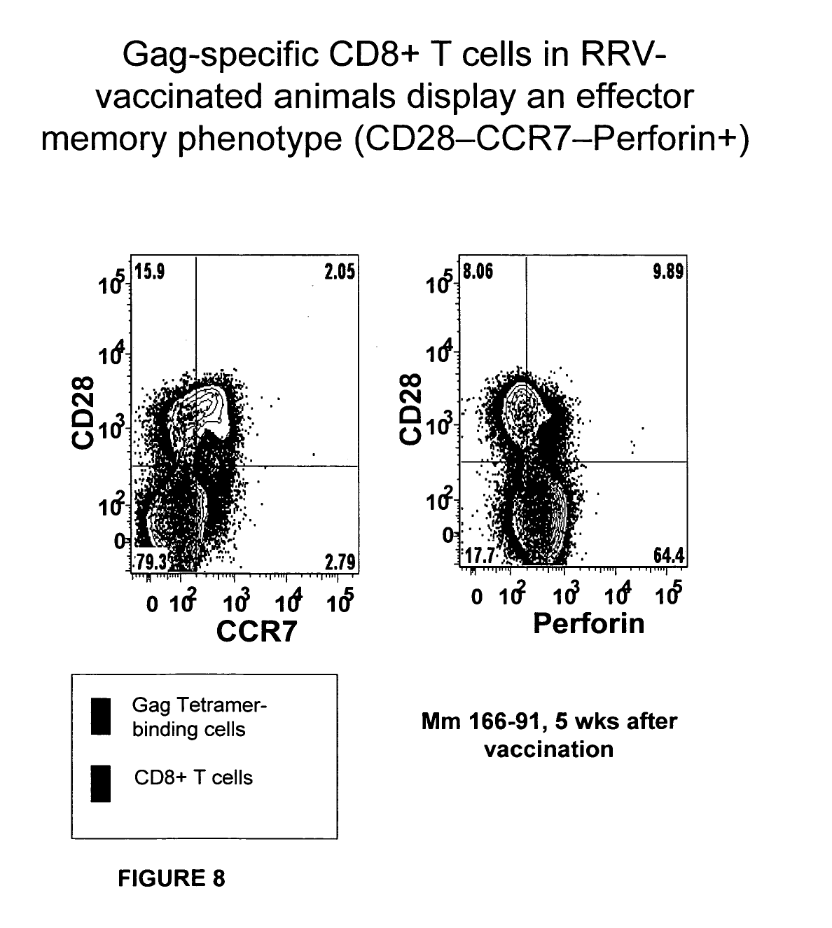

FIG. 9 depicts an AE647 RRV ELISA Timecourse.

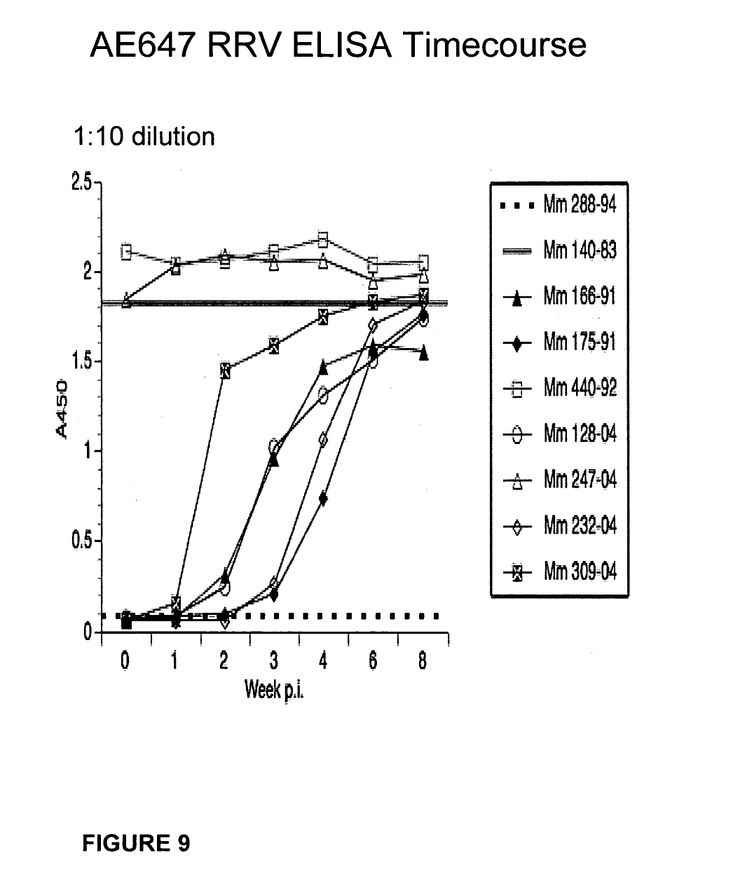

FIG. 10 depicts post challenge viral loads in control and RRV-SIV vaccinated animals.

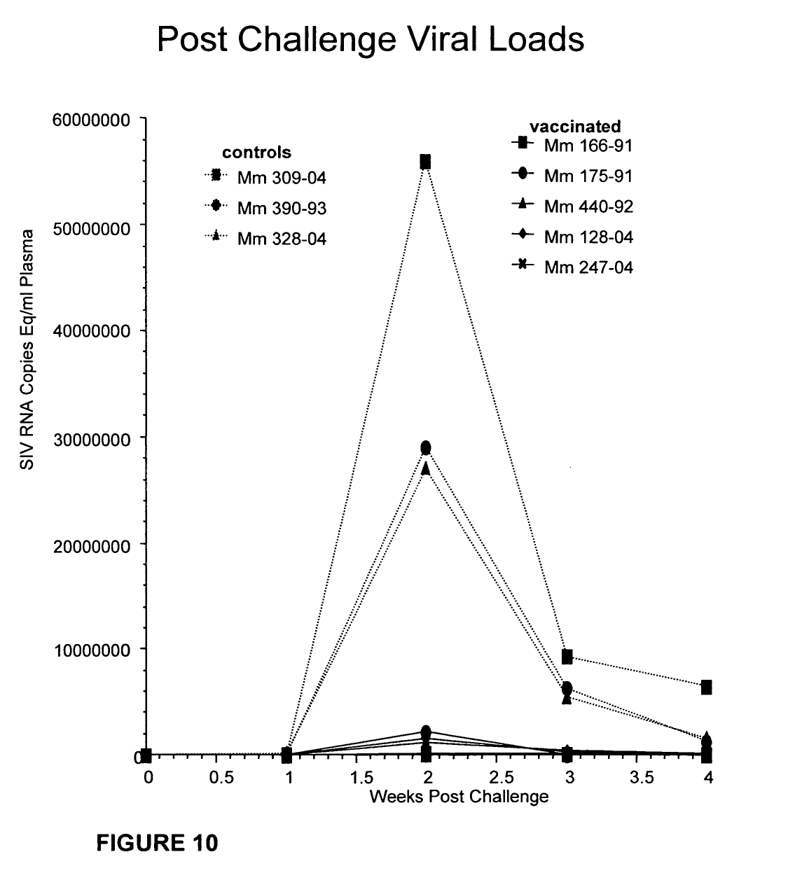

FIG. 11 depicts post challenge anti-SIV Env antibodies.

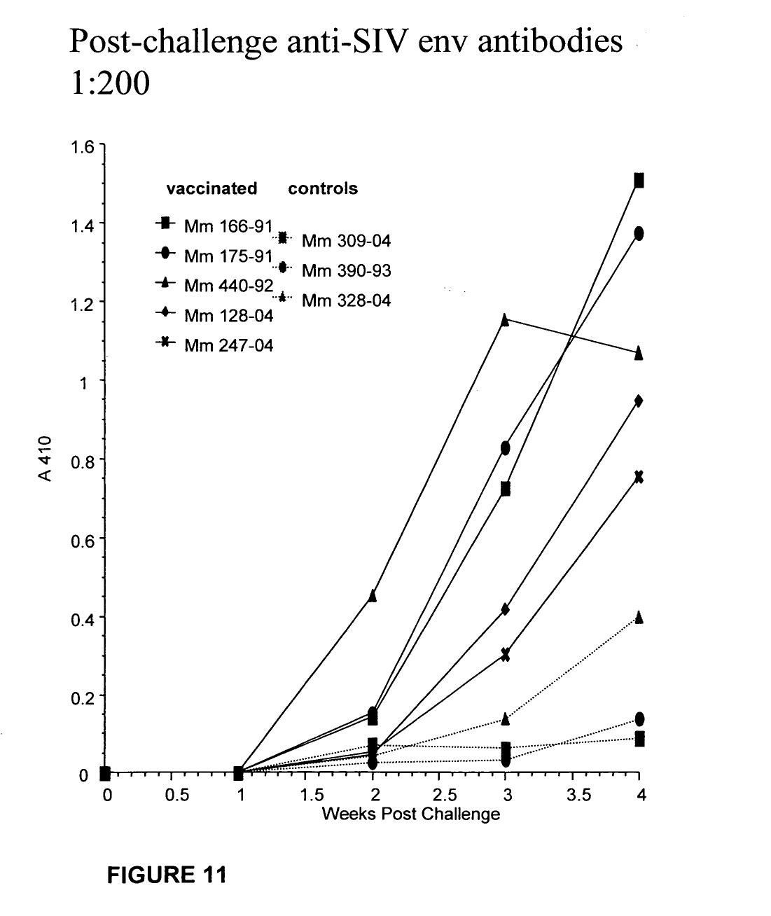

FIG. 12 depicts Env expression in virions (239 mutants).

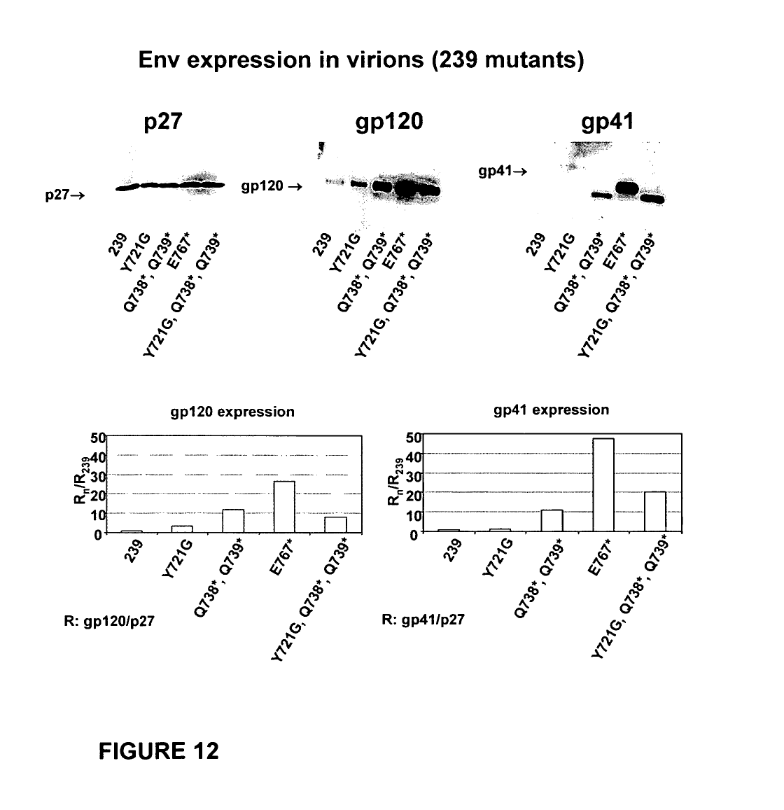

FIG. 13 depicts GP41 mutations. The gp41 cytoplasmic domain in lentiviruses is long compared with retroviruses.

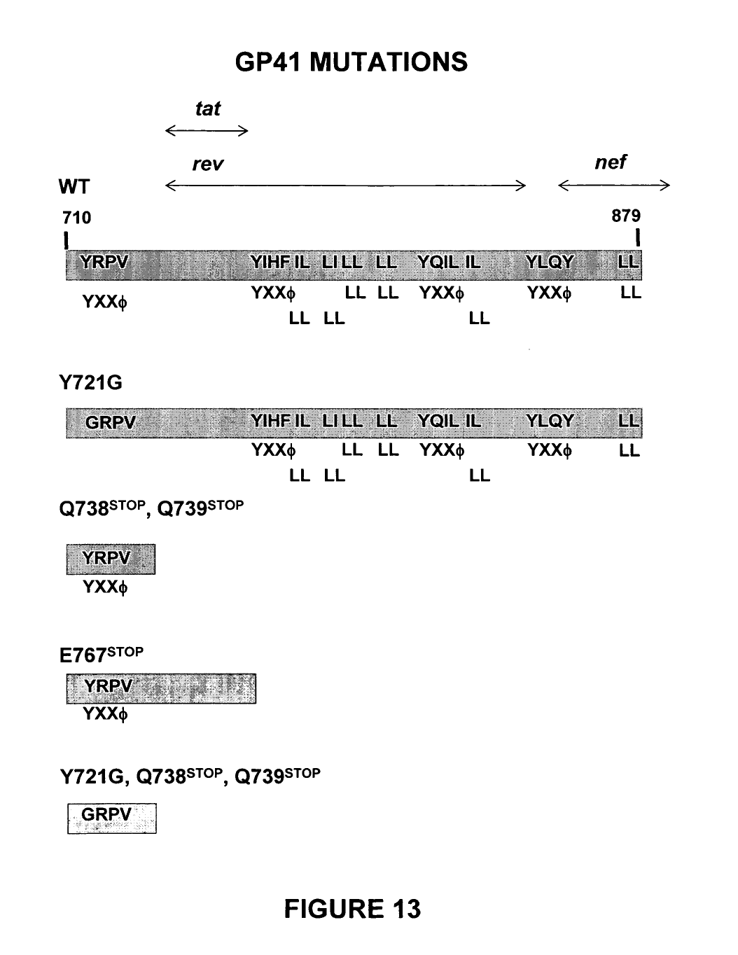

FIG. 14 depicts the expression of various GP41 mutants in cell lysates and culture supernatants. The cell surface expression was analyzed by flow cytometry for each one of the mutants. The mutants with truncated cytoplasmic domains have higher levels of cell surface expression. The highest increase is observed in the E767stop mutant and the lowest increase observed is in the mutant with the tyrosine mutated in the first endocytosis signal.

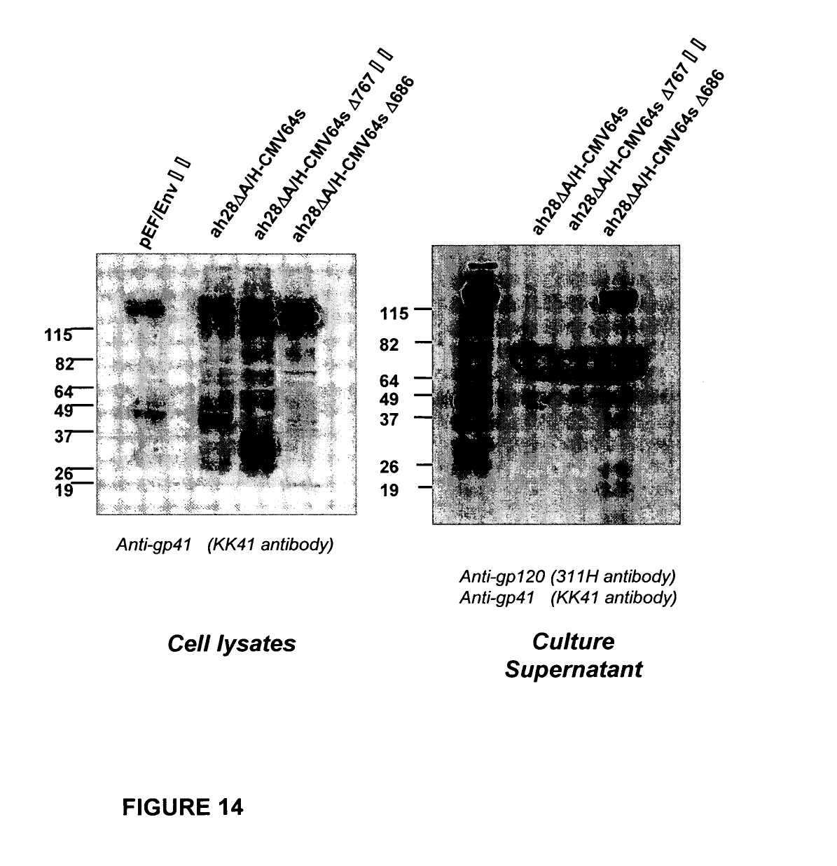

### DETAILED DESCRIPTION

In some aspects, the invention relates to the production and formulation of anti-infective agents. In some aspects, the invention relates to prophylactic or therapeutic methods using virus based delivery vectors to provide a form of immunity (also referred to as sterilizing immunity) that relies on the expression of neutralizing agents to inhibit pathogenic infection or growth. Aspects of the invention are different from traditional immunization techniques that relate to stimulating the immune response of a subject against a pathogen rather than directly inhibiting the pathogen. In some embodiments, the vectors are based on an attenuated virus. In certain embodiments, the vectors are based on the gamma herpes virus. In some embodiments, the gamma herpes virus is a gamma-2 herpes virus (a rhadinovirus), and optionally is an attenuated gamma-2 herpes virus.

In some aspects the invention relates to the delivery of neutralizing agents to a subject to prevent or inhibit infection or disease progression (e.g., viral infection or proliferation) within a subject. In some embodiments, compositions and methods are for the treatment of a subject having, or suspected of having, been exposed to, or infected by, a disease-associated organism. A disease-associated organism may be a pathogenic bacterium, yeast, amoeba, virus, or other pathogenic microorganism. In some embodiments, the viral vectors are for administration to a subject as a prophylactic treatment for an immunodeficiency virus. In some embodiments, the neutralizing agents are specific immunodeficiency virus (e.g., HIV) neutralizing agents. In preferred embodiments, the agents are antibodies (e.g., human or humanized antibodies) that are specific for one or more human immunodeficiency virus (HIV) antigens. However, compositions of the invention may be used to prevent infection or disease progression associated with other viral infections (e.g., papillomavirus, rabies, etc.).

In some aspects, the invention relates to recombinant viruses. As used herein, a recombinant virus is a virus (e.g., a host virus) that is engineered to express a neutralizing agent. Typically, a host virus has integrated in its genome at least one transgene (e.g., one or more transgenes) having at least one coding sequence for a neutralizing agent (e.g., one or more neutralizing agents), that is operatively linked to a promoter and other appropriate regulatory sequences (e.g., enhancer, poly A tail, etc.), wherein the transgene is capable of expressing the at least one neutralizing agent under appropriate culture conditions in vivo or in vitro. In some embodiments, the recombinant virus is formulated in a pharmaceutical preparation for administration to a subject having, or suspected of having, been exposed to, or infected by, a pathogenic organism (e.g., an immunodeficiency virus). In some embodiments, the recombinant virus is formulated as a recombinant pharmaceutical preparation for administration to a subject as a prophylactic treatment for an immunodeficiency virus.

In some aspects, the present invention relates to the discovery that gamma herpes viruses possess a number of advantageous characteristics as a delivery vector (e.g., for preventing and/or treating immunodeficiency viruses such as HIV, SIV, and associated diseases). For example, recombinant gamma herpes viruses have large double-stranded DNA (dsDNA) genomes and, as disclosed herein, can be engineered to express neutralizing agents from one or more transgenes integrated in their genomes. In addition, gamma herpes viruses comprise a diverse set of family members from which to choose an appropriate host virus (e.g., human herpes virus-8, HHV-8) for the delivery of a neutralizing agent to a subject (e.g., a human). Also, gamma herpes viruses target B-cells. Moreover, aspects of the invention are based on the discovery that persistence of gamma herpes viral infections makes the virus particularly useful as a host virus for the delivery of neutralizing agents and provides long term protection against infection and/or prolonged reduction of chronic infection loads (e.g., reduced viral loads in a subject infected by an immunodeficiency virus).

In aspects of the invention, recombinant gamma-2 herpes viruses is used as a prophylactic or therapeutic approach for treating AIDS. In some embodiments, a gamma-2 herpes virus of rhesus monkeys (the rhesus monkey rhadinovirus, RRV, which is closely related to HHV-8) is engineered to express virus neutralizing proteins. In specific embodiments, recombinant RRV provides potent protection against AIDS in monkeys following challenge with the simian immunodeficiency virus (SIV). Thus, the invention in some aspects provides a new therapeutic approach for preventing or treating AIDS in humans. For example, recombinant HHV-8 can be used to protect humans against HIV/AIDS, and/or against other infectious diseases.

### Herpesvirus

In some aspects, the invention relates the use of herpesvirus in recombinant viral vectors. The Herpesviridae are a large family of DNA viruses that cause diseases in humans and animals. Herpes viruses all share a common structure and are composed of relatively large double-stranded, linear DNA genomes encoding 100-200 genes encased within an icosahedral protein cage called the capsid which is itself wrapped in a lipid bilayer membrane called the envelope. This particle is known as the virion. The large genome provides many non-essential sites for introducing one or more transgenes without inactivating the virus (e.g., without completely inhibiting infection or replication). However, it should be appreciated virus vectors of the invention are preferably attenuated so that they do not cause diseases themselves.

All Herpes viruses are nuclear-replicating—the viral DNA is transcribed to RNA within the infected cell's nucleus. Infection is initiated when a viral particle contacts a cell with specific types of receptor molecules on the cell surface. Following binding of viral envelope glycoproteins to cell membrane receptors, the virion is internalized and dismantled, allowing viral DNA to migrate to the cell nucleus. Within the nucleus, replication of viral DNA and transcription of viral genes occurs. Herpes virus is divided into three subfamilies, alpha, beta, and gamma. The human herpes virus classification is outlined in Table 1. Aspects of the invention disclosed here relate to the gamma subfamily of herpesvirus. Herpes virus subfamily gamma is subdivided into four genera that include Lymphocryptovirus, Rhadinovirus, Macavirus, and Percavirus and that are characterized by variable reproductive cycles. Gamma herpesviruses (e.g., Gamma-2 herpesviruses) persist largely in lymphoid tissues, mostly B cells.

TABLE 1
Human Herpes virus (HHV) classification

| Type | Synonym | Subfamily |
|---|---|---|
| HHV-1 | Herpes simplex virus-1 (HSV-1) | Alpha |
| HHV-2 | Herpes simplex virus-2 (HSV-2) | Alpha |
| HHV-3 | Varicella zoster virus (VZV) | Alpha |
| HHV-4 | Epstein-Barr | Gamma |
| | virus (EBV), lymphocryptovirus | |
| HHV-5 | Cytomegalovirus (CMV) | Beta |
| HHV-6, -7 | Roseolovirus | Beta |
| HHV-8 | Kaposi's sarcoma-associated | Gamma |
| | herpesvirus (KSHV), a type of | |
| | rhadinovirus | |

The genus Lymphocryptovirus infects B-cells in humans and new world primates and includes the type species human herpesvirus 4 (HHV-4), also referred to as the Epstein-Barr virus. Other exemplary lymphocryptoviral species include: chimpanzee lymphocryptovirus (Pongine herpesvirus 1, PoHV-1, Herpesvirus pan), orangutan lymphocryptovirus (Pongine herpesvirus 2, PoHV-2, Orangutan herpesvirus), gorilla lymphocryptovirus (Herpesvirus gorilla, Pongine herpesvirus 3, PoHV-3), baboon lymphocryptovirus (baboon herpesvirus, Herpesvirus papio, HVP, Cercopithecine herpesvirus 12, CeHV-12), African green monkey EBV-like virus (Cercopithecine herpesvirus 14, CeHV-14), rhesus lymphocryptovirus (rhesus LCV, RLV, Cercopithecine HV 15), and marmoset lymphocryptovirus (Callitrichine HV 3, Ca1HV-3, CHV3).

The genus Rhadinovirus includes the Human herpesvirus 8 (HHV-8), also known as Kaposi's sarcoma-associated herpesvirus (KSHV), which causes Kaposi's sarcoma, primary effusion lymphoma and multicentric Castleman's disease. Other names for the Rhadinovirus genus include Rhadinoviridae and gamma-2 herpesviruses. They are large double-stranded viruses that possess up to 100 genes in a single long chromosome which is flanked by repetitive DNA sequences called terminal repeats. Rhadinoviruses generally infect B_lymphocytes and fibroblasts and once infection occurs, it is generally life-long (e.g., persistent infection). Rhadinoviruses have been found in New World monkeys such as the squirrel monkeys (herpesvirus saimiri) and in mice (murine gammaherpesvirus-68). More recently, both KSHV-like viruses and a new form of rhadinovirus called rhesus rhadinovirus have been discovered in Old World monkeys. These findings suggest that an additional human tumor virus related to KSHV may be found in humans. Exemplary Rhadinoviruses include: Alcelaphine herpesvirus 1 (AIHV-1), Alcelaphine herpesvirus 2 (AIHV-2), Ateline herpesvirus 2 (AtHV-2), Bovine herpesvirus 4 (BoHV-4), Cercopithecine herpesvirus 17 (CeHV-17), Equid herpesvirus 2 (EHV-2), Equid herpesvirus 5 (EHV-5), Equid herpesvirus 7 (EHV-7), Hippotragine herpesvirus 1 (HiHV-1), Human herpesvirus 8 (HHV-8), Murid herpesvirus 4 (MuHV-4), Ovine herpesvirus 2 (OvHV-2), and Saimiriine herpesvirus 2 (SaHV-2).

In some aspects any of the foregoing gamma herpes virus are appropriate host viruses for the delivery of neutralizing agents. In some cases it may be desirable for the host virus to be selected to match the target subject. For example, if a target subject is a human then an appropriate host virus might be a human herpes virus-8 or a human herpesvirus 4. However, the invention is not so limited.

Human Immunodeficiency Virus

In some aspects, the invention relates the use of neutralizing agents (e.g., antibodies, antibody fragments, aptamers, or other neutralizing protein or nucleic acid agents) that target immunodeficiency virus antigens. As used herein, immunodeficiency virus refers to any one of various strains, subtypes, clades, and stocks of HIV (e.g., HIV-1, HIV-2), SIV and other lentiviruses. Exemplary immunodeficiency viruses are outlined in Table 2.

Immunodeficiency viruses, such as HIV, are members of the genus Lentivirus, which are single-stranded, positive-sense, enveloped RNA viruses. Typically lentiviruses are composed of two copies of positive single-stranded RNA that codes for the virus's genes enclosed by a conical capsid. Table 3 provides a list of exemplary HIV/SIV genes and the functions of their protein products. Of the genes that are encoded within the HIV RNA genome, three of these genes, gag, pol, and env, contain information needed to make the structural proteins for new virus particles. For example, env codes for a protein called gp160 that is broken down by a viral enzyme to form gp120 and gp41. The six remaining genes, tat, rev, nef, vif, vpr, and vpu (or vpx in the case of HIV-2), are regulatory genes for proteins that control the ability of HIV to infect cells, produce new copies of virus (replicate), or cause disease. The protein encoded by nef, for instance, appears necessary for the virus to replicate efficiently, and the vpu-encoded protein influences the release of new virus particles from infected cells. The ends of each strand of HIV RNA contain an RNA sequence called the long terminal repeat (LTR). Regions in the LTR act as switches to control production of new viruses and can be triggered by proteins from either HIV or the host cell. Any one or more of the viral proteins described herein may be targeted by a neutralizing agent of the invention. In specific embodiments, neutralizing agents that target env protein antigens (e.g., amino acid sequences of gp120 or gp41) are used to inhibit pathogenic infection or growth according to the methods disclosed herein.

HIV differs from many viruses in that it has very high genetic variability. This diversity is a result of its fast replication cycle, coupled with a high mutation rate, which leads to the generation of many variants of HIV in a single infected patient. This variability is compounded when a single cell is simultaneously infected by two or more different strains of HIV.

The closely related simian immunodeficiency virus (SIV) exhibits a somewhat different behavior: in its natural hosts, African green monkeys and sooty mangabeys, the retrovirus is present in high levels in the blood, but evokes only a mild immune response, does not cause the development of simian AIDS, and does not undergo the extensive mutation and recombination typical of HIV. By contrast, infection of heterologous hosts (rhesus or cynomologus macaques) with SIV results in the generation of genetic diversity that is on the same order as HIV in infected humans; these heterologous hosts also develop simian AIDS.

Two species of HIV infect humans: HIV-1 and HIV-2. The genetic sequence of HIV-2 is only partially homologous to HIV-1 and more closely resembles that of SIV than HIV-1. HIV-1 is the virus that was initially discovered and termed LAV. It is more virulent, relatively easily transmitted, and is the cause of the majority of HIV infections globally. HIV-2 is less transmittable than HIV-1 and is largely confined to West Africa (Reeves, J. D. et al., J. Gen. Virol. 83 (Pt 6): 1253-1265).

Three groups of HIV-1 have been identified on the basis of differences in env: M, N, and O. Group M is the most prevalent and is subdivided into eight subtypes (or clades), based on the whole genome, which are geographically distinct. The most prevalent are subtypes B (found mainly in North America and Europe), A and D (found mainly in Africa), and C (found mainly in Africa and Asia); these subtypes form branches in the phylogenetic tree representing the lineage of the M group of HIV-1. Coinfection with distinct subtypes gives rise to circulating recombinant forms (CRFs).

TABLE 2
Immunodeficiency Virus
Bovine immunodeficiency virus

| | | |
|---|---|---|
| Bovine immunodeficiency virus | [M32690] | (BIV) |

Equine lentivirus group:

| | | |
|---|---|---|
| Equine infectious anemia virus | [M16575] | (EIAV) |

Feline lentivirus group:

| | | |
|---|---|---|
| Feline immunodeficiency virus (Oma) | [FIU56928] | (FIV-O) |
| Feline immunodeficiency virus (Petuluma) | [M25381] | (FIV-P) |
| Puma lentivirus | [PLU03982] | (PLV-14) |

Ovine/caprine lentivirus group:

| | | |
|---|---|---|
| Caprine arthritis encephalitis virus | [M33677] | (CAEV) |
| Visna/maedi virus (strain 1514) | [M60609] | (VISNA) |
| Visna/maedi virus (strain 1514) | [M60610] | (VISNA) |

Accession

Human immunodeficiency virus 1 (HIV-1)

| | | |
|---|---|---|
| Clade A | | |
| Human immunodeficiency virus 1.U455 | [M62320] | (HIV-1.U455) |
| Clade B | | |
| Human immunodeficiency virus 1.ARV-2/SF-2 | [K02007] | (HIV-1.ARV-2/SF-2) |
| Human immunodeficiency virus 1.BRU (LAI) | [K02013] | (HIV-1.BRU(LAI)) |
| Human immunodeficiency virus 1.HXB2 | [K03455] | (HIV-1.HXB2) |
| Human immunodeficiency virus 1.MN | [M17449] | (HIV-1.MN) |
| Human immunodeficiency virus 1.RF | [M17451] | (HIV-1.RF) |
| Clade C | | |
| Human immunodeficiency virus 1.ETH2220 | [U46016] | (HIV-1.ETH2220) |
| Clade D | | |
| Human immunodeficiency virus 1.ELI | [X04414] | (HIV-1.ELI) |
| Human immunodeficiency virus 1.NDK | [M27323] | (HIV-1.NDK) |
| Clade F | | |
| Human immunodeficiency virus 1.93BR020 | [AF005494] | (HIV-1.93BR020) |
| Clade H | | |
| Human immunodeficiency virus 1.90CR056 | [AF005496] | (HIV-1.90CR056) |
| Clade O | | |
| Human immunodeficiency virus 1.ANT70 | [L20587] | (HIV-1.ANT70) |

Human immunodeficiency virus 2 (HIV-2)

| | | |
|---|---|---|
| Clade A | | |
| Human immunodeficiency virus 2.BEN | [M30502] | (HIV-2.BEN) |
| Human immunodeficiency virus 2.ISY | [J04498] | (HIV-2.ISY) |
| Human immunodeficiency virus 2.ROD | [M15390] | (HIV-2.ROD) |
| Human immunodeficiency virus 2.ST | [M31113] | (HIV-2.ST) |
| Clade B | | |
| Human immunodeficiency virus 2.D205 | [X61240] | (HIV-2.D205) |
| Human immunodeficiency virus 2.EHOA | [U27200] | (HIV-2.EHOA) |
| Human immunodeficiency virus 2.UC1 | [L07625] | (HIV-2.UC1) |

Simian immunodeficiency virus (SIV)

| | | |
|---|---|---|
| African green monkey | | |
| African green monkey 155 | [M29975] | (SIV-agm.155) |
| African green monkey 3 | [M30931] | (SIV-agm.3) |
| African green monkey gr-1 | [M58410] | (SIV-agm.gr) |
| African green monkey Sab-1 | [U04005] | (SIV-agm.sab) |
| African green monkey Tan-1 | [U58991] | (SIV-agm.tan) |
| African green monkey TYO | [X07805] | (SIV-agm.TYO) |
| Simian immunodeficiency virus-chimpanzee | [X52154] | (SIV-cpz) |
| Simian immunodeficiency virus-mandrill | [M27470] | (SIV-mnd) |
| Simian immunodeficiency virus-pig-tailed macaque | [M32741] | (SIV-mne) |
| Simian immunodeficiency virus-red capped | [AF028607] | (SIV-rcm) |
| mangabey | | |
| Simian immunodeficiency virus-Rhesus (Maccaca | [M19499] | (SIV-mac) |
| mulatta) | | |
| Simian immunodeficiency virus-sooty mangabey | [X14307] | (SIV-sm) |
| SIV-H4 | | |
| Simian immunodeficiency virus-stump-tailed | [M83293] | (SIV-stm) |
| macaque | | |
| Simian immunodeficiency virus-sykes monkey | [L06042] | (SIV-syk) |

TABLE 3
HIV/SIV GENES/PROTEINS

| NAME | SIZE | FUNCTION | LOCALIZATION |
|---|---|---|---|
| Gag | | | |
| MA | p17 | membrane anchoring; Env | virion |
| | | interaction; nuclear transport of | |
| | | viral core (myristylated protein | |
| CA | p24 | core capsid | virion |
| NC | p7 | nucleocapsid, binds RNA | virion |
| | p6 | binds Vpr | virion |
| Pol | | | |
| Protease (PR) | p15 | Gag/Pol cleavage and maturation | virion |
| Reverse | p66 | reverse transcription, R Nase H | virion |
| transcriptase (RT), | p51 | activity | |
| RNase H | p15 | | |
| Integrase (IN) | p31 | DNA provirus integration | virion |
| Env | gp120/ | external viral glycoproteins bind | plasma membrane, |
| | gp41 | to CD4 and secondary receptors | virion envelope |
| Tat | p16/p14 | viral transcriptional transactivator | primarily in |
| | | | nucleolus/nucleus |
| Rev | p19 | RNA transport, stability and | primarily in |
| | | utilization factor (phosphoprotein) | nucleolus/nucleus |
| | | | shuttling between |
| | | | nucleolus and |
| | | | cytoplasm |
| Vif | p23 | promotes virion maturation and | cytoplasm (cytosol, |
| | | infectivity | membranes) virion |
| Vpr | p10-15 | promotes nuclear localization of | virion, nucleus |
| | | preintegration complex, inhibits | (probably nuclear |
| | | cell division, arrests infected cells | membrane) |
| | | of G2/M | |
| Vpu | p16 | promotes extracellular release of | integral membrane |
| | | viral particles; degrades CD4 in | protein |
| | | the ER; (phosphoprotein only in | |
| | | HIV-1 and SIVcpz) | |
| Nef | p25-p27 | CD4 and class I downregulation | plasma membrane, |
| | | (myristylated protein) | cytoplasm (probably |
| | | | virion) |
| Vpx | p12-16 | Vpr homolog (not in HIV-1, only in | virion (probably |
| | | HIV-2 and SIV) | nucleus) |

### Recombinant Viruses

The present invention relates to recombinant viral vectors. In particular, it relates to genetically engineered recombinant viruses for use as delivery vectors; pharmaceutical compositions comprising the recombinant viruses; cells for the production of the recombinant viruses; and methods relating to the production of neutralizing agents. In some aspects, the invention relates to recombinant gamma herpes viruses. As used herein, a recombinant gamma herpes virus is a gamma herpes virus (e.g., a host gamma herpes virus) that is engineered to express a heterologous transcript that encodes (or is, e.g., aptamer, shRNA, miRNA) a neutralizing agent. In some embodiments, a host gamma herpes virus has integrated in its genome at least one transgene (e.g., one or more transgenes), wherein the transgene comprises at least one coding region of a neutralizing agent (e.g., one or more coding regions of neutralizing agents). It is to be understood that a coding region may be a nucleic acid sequence that encodes a protein (e.g., an antibody or antibody fragment), or a nucleic acid sequence that corresponds to a functional RNA molecule (e.g., shRNA, miRNA, aptamer) that inhibits the expression or function of a viral protein (e.g., Env protein). In some embodiments, a transgene comprises a coding region of a neutralizing agent that is operatively joined to a promoter and other appropriate regulatory sequences (e.g., enhancer, poly A tail, etc.), such that the transgene is capable of expressing the neutralizing agent under appropriate culture conditions in vivo or in vitro. In some embodiments, a transgene comprises a plurality of coding regions of neutralizing agents that are each operatively joined to a promoter and other appropriate regulatory sequences (e.g., enhancer, poly A tail, etc.), such that the transgene is capable of expressing a plurality of neutralizing agents under appropriate culture conditions in vivo or in vitro. In some embodiments, a transgene comprises a plurality of coding regions of neutralizing agents that are each operatively joined to a common promoter and other appropriate regulatory sequences (e.g., enhancer, poly A tail, etc.), such that the transgene is capable of expressing a polypeptide consisting of a plurality of neutralizing agents (e.g., a fusion polypeptide) under appropriate culture conditions in vivo or in vitro. In some embodiments, the trangenes encode neutralizing agents (e.g., antibodies, antibody fragments, aptamers, etc.) that target immunodeficiency virus proteins such as, for example, Gag, Rev-Tat-Nef, and/or Env. In some embodiments, the coding sequence of the transgene is operably linked to a CMVie, SV40, or EF1 promoter.

As used herein, a coding sequence (e.g., the coding sequence of a neutralizing agent) and regulatory sequences are said to be “operably” joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript might be translated into the desired protein or polypeptide. Similarly two or more coding regions are operably joined when they are linked in such a way that their transcription from a common promoter results in the expression of two or more proteins having been translated in frame. In some embodiments, operably joined coding sequences may have internal ribosomal entry sites (IRES) between them and thereby produce separate proteins from a common transcript. In some embodiments, operably joined coding sequences yield a fusion protein.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. Especially, such 5′ non-transcribed regulatory sequences will include a promoter region that includes a promoter sequence for transcriptional control of the operably joined gene. Regulatory sequences may also include enhancer sequences or upstream activator sequences as desired. The vectors of the invention may optionally include 5′ leader or signal sequences. The choice and design of an appropriate vector is within the ability and discretion of one of ordinary skill in the art.

In some embodiments, a transgene is prepared by obtaining the coding region, or fragment thereof, and optionally the promoter and other regulatory regions, of an immunodeficiency virus neutralizing agent (e.g., antigen binding agent). In some embodiments, the coding region, or fragment thereof, of an immunodeficiency neutralizing agent (e.g., antigen binding agent) is cloned into a subcloning plasmid. In some embodiments, the coding region or fragment thereof is cloned into a subcloning plasmid that is an expression vector, which is a plasmid vector into which a desired DNA sequence may be inserted by restriction and ligation such that the DNA sequence is operably joined to regulatory sequences (e.g., a promoter). The subcloning plasmids may further contain one or more marker sequences, which may or may not be operably linked to the coding sequence. Marker sequences include, for example, those encoding proteins that increase or decrease either resistance or sensitivity to antibiotics or other compounds, genes that encode enzymes whose activities are detectable by standard assays known in the art (e.g., β-galactosidase or alkaline phosphatase), and fluorescent protein (e.g., green fluorescent protein). Marker sequences can also be affinity tags such as 6-His and others that are well known in the art. As used herein, subcloning plasmids (including expression vectors) having coding regions or fragments thereof of an immunodeficiency virus neutralizing agent (e.g., antigen binding agent) are referred to as subclones.

In some embodiments, the coding region, or fragment thereof, of an immunodeficiency virus neutralizing agent (e.g., antigen binding agent) is cloned directly into a large cloning vector (e.g., Cosmids, a Bacterial Artificial Chromosome, a Yeast Artificial Chromosome) that contains a recombinant viral genome or a portion thereof. In some embodiments, the coding region, or fragment thereof, of an immunodeficiency virus neutralizing agent (e.g., antigen binding agent) is cloned directly into a viral genome.

Methods for cloning a transgene (e.g., from a subclone) into a recombinant viral genome are disclosed herein and are known in the art, as exemplified in Bilello J P, Journal of Virology, February 2006, p. 1549-1562, Vol. 80, No. 3. In some embodiments, cloning of a transgene into a gamma herpes virus genome comprises constructing a genomic library (e.g., a cosmid library comprising the gamma herpes virus genome).

In some embodiments, a site for inserting the transgene into the viral genome is selected a priori and a specific cloning strategy is used to incorporate the transgene into the viral genome. Appropriate cloning strategies are well known in the art and are exemplified herein. In some embodiments, homologous recombination techniques are used. However, a priori selection of the incorporation site is not necessary and random gene integration can be used provided that the transgene remains operably linked to any necessary promoter and regulatory elements and, typically, provided that the site of integration does not abrogate an essential endogenous viral gene.

In some embodiments, the insertion site is upstream from R1 promoter. In some embodiments, a transgene encoding a neutralizing agent (e.g., a neutralizing agent that targets Env) is integrated near terminal repeat regions.

In some embodiments, the transgene is integrated into a non-essential viral gene and, optionally, its coding region is operably linked the endogenous promoter of the non-essential viral gene. In some embodiments, the transgene is integrated into a non-essential viral gene and, optionally, its coding region is operably linked to a constitutive transgenic promoter. In some embodiments, the transgene is integrated into a non-coding region of the viral genome and, optionally its coding region is operably linked to a constitutive transgenic promoter. Suitable constitutive transgenic promoters are disclosed herein (e.g., CMV) and are well known in the art. In some embodiments, the transgene is integrated into the viral genome (e.g., in a non-essential gene) and, optionally its coding region is linked to an inducible promoter. Suitable inducible promoters are well known in the art, such as a tetracycline inducible promoter.

In some embodiments, the transgene is integrated into the host virus genome such that its coding region is operably linked to the coding region of an endogenous gene. In some embodiments, a transgene operably linked to an endogenous gene may have an internal ribosomal entry site between its coding region and the coding region of an endogenous gene and, thus, encode one or more separate proteins that are translated from the same mRNA transcript. In some embodiments, a transgene's coding region that is operably linked to the coding region of an endogenous gene may encode a fusion protein consisting of the endogenous gene product and the transgene product. Such fusion proteins may have the transgene product (e.g., a neutralizing agent) fused at the N- and/or C-Terminus of the endogenous gene product.

In some embodiments, a transgene is integrated into the viral genome in such a way that the virus becomes attenuated. For example, a transgene may be integrated into a non-essential gene of the host virus or the promoters or other regulatory regions of one or more non-essential genes of the host virus, thereby inactivating the non-essential genes, which causes the host virus to become attenuated.

In some aspects, the invention relates to live attenuated viruses, which are viruses that have been rendered less pathogenic to the host, either by specific genetic manipulation of the virus genome, or by passage in some type of tissue culture system.

In some cases, live attenuated viruses have been made by deleting an non-essential gene or genetically altering one or more essential genes such that the genes are still functional, but do not operate completely effectively.

In some aspects, the invention relates to live attenuated gamma-2-herpesviruses. In some embodiments, attenuation is achieved by deletion of a non-essential gamma-2-herpesvirus gene. In some embodiments, the non-essential gene is an oncogene. In some embodiments, the oncogene is associated with Kaposi's sarcoma. The human herpesvirus 8 (HHV-8) genome consists of a long unique region (140.5 kb) encoding for over 80 open reading frames (ORFs), surrounded by terminal repeat regions (TRs) consisting of 801 base pair direct repeat units with a high G+C content. Three large regions contain genes conserved among the Rhadinoviruses, whereas the regions between them contain unique genes. Many of these unique genes encode homologues for host cellular proteins. Genes that are potentially important in the pathogenesis of Kaposi Sarcoma are include: CCP, complement control protein; v-cyc, viral D-type cyclin; vFLIP, viral FLICE inhibitory protein; vGPCR, viral G-protein-coupled receptor; vIL-6, viral interleukin 6; vIRF, viral interferon regulatory factor; LANA, latency-associated nuclear antigen; and vMIP, viral macrophage inflammatory protein. Thus, in some embodiments the virus is attenuated by deleting a gene selected from: CCP, v-cyc, vFLIP, vGPCR, vIL-6, vIRF, LANA, and vMIP.

In some aspects, the invention relates to the use of live attenuated viruses that are recombinant vectors for delivering one or more neutralizing agents. In some embodiments, aspects of the invention also relate to immunizing a subject with one or more antigens in addition to providing one or more neutralizing agents. An antigen, as used herein, refers to a molecule containing one or more epitopes (either linear, conformational or both) that will stimulate a host's immune system to make a humoral and/or cellular antigen-specific response. The term is used interchangeably with the term immunogen. Normally, a B-cell epitope will include at least about 5 amino acids but can be as small as 3-4 amino acids. A T-cell epitope, such as a CTL epitope, will include at least about 7-9 amino acids, and a helper T-cell epitope at least about 12-20 amino acids. Normally, an epitope will include between about 7 and 15 amino acids, such as, 9, 10, 12 or 15 amino acids.

Furthermore, for purposes of the present invention, an antigen refers to a protein that includes modifications, such as deletions, additions and substitutions (generally conservative in nature), to the native sequence, so long as the protein maintains the ability to elicit an immunological response, as defined herein. These modifications may be deliberate, as through site-directed mutagenesis, or may be accidental, such as through mutations of hosts that produce the antigens.

In some embodiments, the coding region or fragment thereof, and optionally the associated promoter and other regulatory regions, of a immunodeficiency virus antigen are obtained from a immunodeficiency virus sample that was obtained from a subject. In some embodiments, a virus sample is obtained from a biological specimen from the subject. As used herein, a biological specimen includes, but is not limited to: tissue, cells and/or body fluid (e.g., serum, blood, lymph node fluid, etc.). The biological specimen may include cells and/or fluid. The tissue and cells may be obtained from a subject or may be grown in culture (e.g. from a cell line). As used herein, a biological specimen is body fluid, tissue or cells obtained from a subject using methods well-known to those of ordinary skill in the related medical arts. In some embodiments, the coding region or fragment thereof, and optionally the associated promoter and other regulatory regions, of a immunodeficiency virus antigen that is obtained from a immunodeficiency virus sample is cloned into a subcloning plamid. In some embodiments, the coding region or fragment thereof, and optionally the associated promoter and other regulatory regions, of a immunodeficiency virus antigen that is obtained from a immunodeficiency virus sample is cloned directly into a recombinant viral genome (e.g., a viral genome comprising a transgene that encodes a neutralizing agent).

In some embodiments, the sequence of the coding region or fragment thereof, of a immunodeficiency virus antigen is modified. In some embodiments, modification of the sequence of coding region or fragment thereof results variants of immunodeficiency virus antigens. The skilled artisan will realize that conservative amino acid substitutions may be made in immunodeficiency virus antigens to provide functionally equivalent variants, or homologs of the foregoing polypeptides, e.g., the variants retain the functional capabilities of the immunodeficiency virus antigen (e.g., immunogenicity, reduction of viral load in a subject). In some aspects, the invention embraces sequence alterations that result in conservative amino acid substitution of immunodeficiency virus antigens. As used herein, a conservative amino acid substitution refers to an amino acid substitution that does not alter the relative charge or size characteristics of the protein in which the amino acid substitution is made. Variants can be prepared according to methods for altering polypeptide sequence known to one of ordinary skill in the art such as are found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Exemplary functionally equivalent variants or homologs of the immunodeficiency virus antigen include conservative amino acid substitutions of in the amino acid sequences of proteins disclosed herein. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D. Therefore, one can make conservative amino acid substitutions to the amino acid sequence of the immunodeficiency virus antigen disclosed herein and retain the immunological properties such as antibody-binding characteristics.

In some cases, upon determining that a peptide derived from an immunodeficiency virus antigen is presented by an MHC molecule and recognized by antibodies or T lymphocytes (e.g., helper T cells or CTLs), one can make conservative amino acid substitutions to the amino acid sequence of the peptide, particularly at residues which are thought not to be direct contact points with the MHC molecule. For example, methods for identifying functional variants of HLA class II binding peptides are provided in a published PCT application of Strominger and Wucherpfennig (PCT/US96/03182). Peptides bearing one or more amino acid substitutions also can be tested for concordance with known HLA/MHC motifs prior to synthesis using, e.g. the computer program described by D'Amaro and Drijfhout (D'Amaro et al., Human Immunol. 43:13-18, 1995; Drijfhout et al., Human Immunol. 43:1-12, 1995). The substituted peptides can then be tested for binding to the MHC molecule and recognition by antibodies or T lymphocytes when bound to MHC. These variants can be tested for improved stability and are useful, inter alia, in vaccine compositions such as the vaccine compositions used in the boosting regimes disclosed herein.

Conservative amino-acid substitutions in the amino acid sequence of an immunodeficiency virus antigen to produce functionally equivalent variants of an immunodeficiency virus antigen typically are made by alteration of a nucleic acid encoding an immunodeficiency virus antigen. Such substitutions can be made by a variety of methods known to one of ordinary skill in the art. For example, amino acid substitutions may be made by PCR-directed mutation, site-directed mutagenesis according to the method of Kunkel (Kunkel, Proc. Nat. Acad. Sci. U.S.A. 82: 488-492, 1985), or by chemical synthesis of a gene encoding a polypeptide of an immunodeficiency virus antigen. Where amino acid substitutions are made to a small unique fragment of an immunodeficiency virus antigen, such as an antigenic epitope recognized by autologous or allogeneic sera or T lymphocytes, the substitutions can be made by directly synthesizing the peptide. The activity of functionally equivalent variants of immunodeficiency virus antigens can be tested by cloning the gene encoding the altered immunodeficiency virus antigen into a bacterial or mammalian expression vector, introducing the vector into an appropriate host cell, expressing the altered polypeptide, and testing for a functional capability of the immunodeficiency virus antigen. Peptides that are chemically synthesized can be tested directly for function, e.g., for binding to antisera recognizing associated antigens.

Accordingly, in some aspects the invention relates to live attenuated viruses that are recombinant vaccine vectors for heterologous antigens in addition to live attenuated viruses that are vectors for delivering one or more neutralizing agents. It should be appreciated that the individual virus variants that may be isolated for generating subject-specific antigens as described above also may be used for generating specific antibodies that can be used as neutralizing agents as described herein.

An immunological response to an antigen or composition is the development in a subject of a humoral and/or a cellular immune response to an antigen present in the composition of interest. A humoral immune response refers to an immune response mediated by antibody molecules, while a cellular immune response is one mediated by T-lymphocytes and/or other white blood cells. One aspect of cellular immunity involves an antigen-specific response by cytolytic T-cells (CTLs). CTLs have specificity for peptide antigens that are presented in association with proteins encoded by the major histocompatibility complex (MHC) and expressed on the surfaces of cells. CTLs help induce and promote the destruction of intracellular microbes, or the lysis of cells infected with such microbes. Another aspect of cellular immunity involves an antigen-specific response by helper T-cells. Helper T-cells act to help stimulate the function, and focus the activity of, specific effector cells, such as B and plasma cells as well as cytotoxic T cells, against cells displaying peptide antigens in association with MI-IC molecules on their surface. A cellular immune response also refers to the production of cytokines, chemokines and other such molecules produced by activated T-cells and/or other white blood cells, including those derived from CD4+ and CD8+ T-cells. In addition, a chemokine response may be induced by various white blood or endothelial cells in response to an administered antigen.

A composition or vaccine that elicits a cellular immune response may serve to sensitize a subject by the presentation of antigen in association with MHC molecules at the cell surface. The cell-mediated immune response is directed at, or near, cells presenting antigen at their surface. In addition, antigen-specific T-lymphocytes can be generated to allow for the future protection of an immunized host.

The ability of a particular antigen to stimulate a cell-mediated immunological response may be determined by a number of assays, such as by lymphoproliferation (lymphocyte activation) assays, CTL cytotoxic cell assays, or by assaying for T-lymphocytes specific for the antigen in a sensitized subject. Such assays are well known in the art. See, e.g., Erickson et al., J. Immunol. (1993) 151: 4189-4199; Doe et al., Eur. J. Immunol. (1994) 24: 2369-2376. Recent methods of measuring cell-mediated immune response include measurement of intracellular cytokines or cytokine secretion by T-cell populations (e.g., by ELISPOT technique), or by measurement of epitope specific T-cells (e.g., by the tetramer technique) (reviewed by McMichael, A. J., and O'Callaghan, C. A., J. Exp. Med. 187 (9): 1367-1371, 1998; Mcheyzer-Williams, M. G., et al, Immunol. Rev. 150: 5-21, 1996; Lalvani, A., et al, J. Exp. Med. 186: 859-865, 1997).

Thus, an immunological response as used herein may be one that stimulates the production of CTLs, and/or the production or activation of helper T-cells. The production of chemokines and/or cytokines may also be stimulated. The antigen of interest may also elicit an antibody-mediated immune response. Hence, an immunological response may include one or more of the following effects: the production of antibodies by B-cells; and/or the activation of suppressor, cytotoxic, or helper T-cells and/or T-cells directed specifically to an antigen or antigens present in the composition or vaccine of interest. These responses may serve to neutralize infectivity, and/or mediate antibody-complement, or antibody dependent cell cytotoxicity (ADCC) to provide protection to an immunized host. Such responses can be determined using standard immunoassays and neutralization assays, well known in the art.

The antigens used in this invention comprise antigens derived from HIV. Such antigens include, for instance, the structural proteins of HIV, such as Env, Gag and Pol. In some embodiments, the antigens of this invention comprise an HIV Env protein, such as gp140. However the invention is not so limited and polypeptide or fragment thereof encoded by an immunodeficiency virus gene can be a suitable antigen (see Table 3). The genes of HIV are located in the central region of the proviral DNA and encode at least nine proteins divided into three major classes: (1) the major structural proteins, Gag, Pol, and Env; (2) the regulatory proteins, Tat and Rev and (3) the accessory proteins, Vpu, Vpr, Vif, and Nef. Many variants are known in the art, including from HIV-1 strains and diverse subtypes (e.g., subtypes, A through G, and O), HIV-2 strains and diverse subtypes, and simian immunodeficiency virus (SIV). (See, e.g., Virology, 3rd Edition (W. K. Joklik ed. 1988); Fundamental Virology, 2nd Edition (B. N. Fields and D. M. Knipe, eds. 1991); Virology, 3rd Edition (Fields, B N, D M Knipe, P M Howley, Editors, 1996, Lippincott-Raven, Philadelphia, Pa.; for a description of these and other related viruses). Any of the proteins and fusions or fragments thereof that are disclosed herein are suitable antigens for use with the recombinant vaccines disclosed herein.

In addition, due to the large immunological variability that is found in different geographic regions for the open reading frame of HIV, particular combinations of antigens may be preferred for administration in particular geographic regions. Briefly, at least eight different subtypes of HIV have been identified and, of these, subtype B viruses are more prevalent in North America, Latin America and the Caribbean, Europe, Japan and Australia.

Almost every subtype is present in sub-Saharan Africa, with subtypes A and D predominating in central and eastern Africa, and subtype C in southern Africa.

Subtype C is also prevalent in India and it has been recently identified in southern Brazil. Subtype E was initially identified in Thailand, and is also present in the Central African Republic. Subtype F was initially described in Brazil and in Romania. The most recent subtypes described are G, found in Russia and Gabon, and subtype H, found in Zaire and in Cameroon. Group O viruses have been identified in Cameroon and also in Gabon. Thus, as will be evident to one of ordinary skill in the art, it is generally preferred to select an HIV antigen that is appropriate to the particular HIV subtype that is prevalent in the geographical region of administration or known to be associated with a particular subject under treatment (e.g., a subtype by which a subject is known to be infected). Subtypes of a particular region may be determined by two-dimensional double immunodiffusion or, by sequencing the HIV genome (or fragments thereof) isolated from individuals within that region.

As utilized herein, immunogenic portion refers to a portion of the respective antigen that is capable, under the appropriate conditions, of causing an immune response (i.e., cell-mediated or humoral).

The immunogenic portion(s) used for immunization may be of varying length, although it is generally preferred that the portions be at least 9 amino acids long and may include the entire antigen. Immunogenicity of a particular sequence is often difficult to predict, although T cell epitopes may be predicted utilizing computer algorithms such as TSITES (Medlmmune, Md.), in order to scan coding regions for potential T-helper sites and CTL sites. From this analysis, peptides are synthesized and used as targets in an in vitro cytotoxic assay. Other assays, however, may also be utilized, including, for example, ELISA, or ELISPOT, which detects the presence of antibodies against the newly introduced vector, as well as assays which test for T helper cells, such as gamma-interferon assays, IL-2 production assays and proliferation assays.

Immunogenic portions may also be selected by other methods. For example, the HLA A2.1 transgenic mouse has been shown to be useful as a model for human T-cell recognition of viral antigens. Briefly, in the influenza and hepatitis B viral systems, the murine T cell receptor repertoire recognizes the same antigenic determinants recognized by human T cells. In both systems, the CTL response generated in the HLA A2.1 transgenic mouse is directed toward virtually the same epitope as those recognized by human CTLs of the HLA A2.1 haplotype (Vitiello et al. (1991) J. Exp. Med. 173: 1007-1015; Vitiello et al. (1992) Abstract of Molecular Biology of Hepatitis B Virus Symposia).

Additional immunogenic portions of the HIV antigens described herein may be obtained by truncating the coding sequence at various locations including, for example, to include one or more epitopes from the various domains of the HIV genome. As noted above, such domains include structural domains such as Gag, Gag-polymerase, Gag-protease, reverse transcriptase (RT), integrase (IN) and Env. The structural domains are often further subdivided into polypeptides, for example, p55, p24, p6 (Gag); p160, p10, p15, p31, p65 (pol, prot, RT and IN); and gp160, gp120 and gp41 (Env). Additional epitopes of HIV and other immunodeficiency virus related diseases are known or can be readily determined using methods known in the art.

It should be appreciated that the antigens described herein may be administered using viral vectors that are similar to those used to deliver one or more neutralizing agents. It also should be appreciated that the type of neutralizing agent used may be optimized depending on the population or geographic region where treatment is performed as described above for the antigens. For example, antibodies that are specific for certain HIV variant epitopes may be used for treating populations that tend to express those epitopes at higher frequencies. However, in some embodiments, neutralizing agents of the invention (e.g., antibodies) are broad range and recognize and/or interfere with a wide range of different viral variants (e.g., HIV variants).

### Virus Preparation

Recombinant herpes virus vectors may be prepared using methods known to one of ordinary skill in the art. For example, methods for the preparation of recombinant gamma herpes virus (e.g., HHV-8) are well known in the art and are exemplified herein. In some embodiments, preparation of recombinant herpes virus comprises the construction of a gamma herpes virus genomic plasmid library (e.g., a cosmid library) from viral genomic DNA. Library production methods are known in the art, for example by using the SuperCos 1 Cosmid Vector Kit from Stratagene, CopyControl™ Fosmid Library Production Kit from EPICENTRE Biotechnologies; EpiFOS™ Fosmid Library Production Kit from EPICENTRE Biotechnologies; pWEB::TNC™ Cosmid Cloning Kit from EPICENTRE Biotechnologies; BigEasy v2.0 Linear Cloning System from Lucigen; and CopyRight v2.0 pSMART BAC BamHI Cloning Kit from Lucigen. Other appropriate library production kits and methods will be apparent to one of ordinary skill in the art. Typically, library plasmids, such as cosmids, that contain large DNA inserts, are sequenced to identify the boundaries of the selected clones. In some embodiments, a series of plasmids (e.g., cosmids) encompassing the entire viral genome, including the terminal repeat regions, are selected with this procedure. Typically, one or more transgenes are cloned into a viral genomic region contained in one or more of the plasmids using methods known in the art and disclosed herein.

In some embodiments, recombinant viruses are prepared by transfecting overlapping library constituents plasmids that comprises the entire viral genome and that include one or more plasmids having a transgene encoding a neutralizing agent (e.g., a immunodeficiency virus antigen binding protein) into a euykarytoic cells (e.g, 293T cells). After about 1 to 10 days, preferably about 5, post-transfection, culture supernatant containing virus are collected and stored as recombinant stocks. Recombinant virus stocks are typically stored between about −80 to about 4° C. Recombinant virus stocks are amplified by infection of target cells. In some embodiments, the target cells are fibroblasts of the species that the host virus targets. For example, HHV-8 virus can be amplified by infection of human fibroblast (e.g., human foreskin fibroblasts, HF cells). In some embodiments, RRV is amplified by infection of rhesus monkey foreskin fibroblasts (RF cells). Typically target cell cultures (e.g., HF cells, RF cells) are inoculated with an aliqout of the recombinant viral stock and are passaged until the emergence of viral plaques is observed in the cultures, and then cultures are typically maintained without splitting until complete lysis of the target cell monolayer. For each recombinant gamma herpes virus, supernatants collected following complete lysis of infected target cells are typically centrifuged to remove cellular debris. The supernatant is typically then filtered (e.g., using filter with a pore size less than about 0.5 um) to remove any additional debris. The filtered supernatant is typically centrifuged at high speed (e.g., more than 15,000 g for more than 1 hour) to pellet the viral particles. Pelleted viral particles can be subsequently resuspended in an aqueous solution and/or used in the preparation of a recombinant viral vector formulations.

In some embodiments, a virus variant that is not highly immunogenic may be used as a vector to minimize the host response against the recombinant virus and thereby promote prolonged presence of the virus in the host subject.

### Viral Vector Formulation

The vectors of the invention may comprise a pharmaceutical composition comprising a recombinant herpes virus (e.g., gamma herpes virus) alone, or in combination with one or more other viruses (e.g., a second recombinant gamma herpes virus encoding one or more different neutralizing agents), and/or in combination with one or more other therapeutic (e.g., immunotherapeutic) agents. In some embodiments, a vaccine comprises 1, 2, 3, 4, 5, 6, 7, 8, 9, 10, or more different recombinant gamma herpes viruses each encoding one or more different neutralizing agents.

Optionally, vaccines and/or immunotherapeutic agents may be added to the vector compositions. As used here, an immunotherapeutic agents refers to a molecule, for example a protein that is capable of modulating an immune response. Non-limiting examples of immunotherapeutic agents include lymphokines (also known as cytokines), such as IL-6, TGF-P, IL-1, IL-2, IL-3, etc.); and chemokines (e.g., secreted proteins such as macrophage inhibiting factor).

Certain cytokines, for example TRANCE, flt-3L, and a secreted form of CD40L are capable of enhancing the immunostimulatory capacity of APCs.

Non-limiting examples of cytokines which may be used alone or in combination in the practice of the present invention include, interleukin-2 (IL-2), stem cell factor (SCF), interleukin 3 (IL-3), interleukin 6 (IL-6), interleukin 12 (IL-12), G-CSF, granulocyte macrophage-colony stimulating factor (GM-CSF), interleukin-1 alpha (IL-1a), interleukin-11 (IL-11), MIP-ly, leukemia inhibitory factor (LIF), c-kit ligand, thrombopoietin (TPO), CD40 ligand (CD40L), tumor necrosis factor-related activation-induced cytokine (TRANCE) and flt3 ligand (flt-3L). Cytokines are commercially available from several vendors such as, for example, Genzyme (Framingham, Mass.), Amgen (Thousand Oaks, Calif.), R&D Systems and Immunex (Seattle, Wash.).

The sequences of many of these molecules are also available, for example, from the GenBank database. It is intended, although not always explicitly stated, that molecules having similar biological activity as wild-type or purified cytokines (e.g., recombinantly produced or mutants thereof) and nucleic acid encoding these molecules are intended to be used within the spirit and scope of the invention.

The compositions of the invention will typically be formulated with pharmaceutically acceptable carriers or diluents. As used herein, the term “pharmaceutically acceptable carrier” refers to a carrier for administration of the recombinant viruses which does not itself induce the production of antibodies harmful to the individual receiving the composition, and which may be administered without undue toxicity. Suitable carriers may be large, slowly metabolized macromolecules such as proteins, polysaccharides, polylactic acids, polyglycolic acids, polymeric amino acids, amino acid copolymers, and inactive virus particles. Examples of particulate carriers include those derived from polymethyl methacrylate polymers, as well as microparticles derived from poly (lactides) and poly (lactide-co-glycolides), known as PLG. See, e.g., Jeffery et al., Pharm. Res. (1993) 10: 362-368; McGee et al. (1997) J Microencapsul. 14 (2): 197-210; O'Hagan et al. (1993) Vaccine 11 (2): 149-54.

Such carriers are well known to those of ordinary skill in the art. Additionally, these carriers may function as immunostimulating agents (“adjuvants”).

Furthermore, compositions of the invention may be conjugated to a bacterial toxoid, such as toxoid from diphtheria, tetanus, cholera, etc., as well as toxins derived from E. coli.

Pharmaceutically acceptable salts can be used therein, for example, mineral acid salts such as hydrochlorides, hydrobromides, phosphates, sulfates, and the like; and the salts of organic acids such as acetates, propionates, malonates, benzoates, and the like. A thorough discussion of acceptable excipients is available in the well-known Remington's Pharmaceutical Sciences.

Pharmaceutically acceptable carriers in therapeutic compositions may contain liquids such as water, saline, glycerol and ethanol. Additionally, auxiliary substances, such as wetting or emulsifying agents, pH buffering substances, and the like, may be present in such vehicles.

By pharmaceutically acceptable or pharmacologically acceptable is meant a material which is not biologically or otherwise undesirable, e.g., the material may be administered to an individual in a formulation or composition without causing any undesirable biological effects or interacting in a deleterious manner with any of the components of the composition in which it is contained.

Further, the compositions described herein can include various excipients, adjuvants, carriers, auxiliary substances, modulating agents, and the like. Preferably, the compositions will include an amount of the neutralizing agent sufficient to prevent infection and or disease progression. An appropriate effective amount can be determined by one of skill in the art. In some embodiments, a dose of more than 10⁷, 10⁸, or 10⁹ recombinant viral particles (e.g., colony forming units) is administered to a subject.

Additional adjuvants may also be used in the invention. Such adjuvants include, but are not limited to: (1) cytokines, such as interleukins (IL-1, IL-2, etc.), macrophage colony stimulating factor (M-CSF), tumor necrosis factor (TNF), beta chemokines (MIP, 1-alpha, 1-beta Rantes, etc.); (2) detoxified mutants of a bacterial ADP-ribosylating toxin such as a cholera toxin (CT), a pertussis toxin (PT), or an E. coli heat-labile toxin (LT), particularly LT-K63 (where lysine is substituted for the wild-type amino acid at position 63) LT-R72 (where arginine is substituted for the wild-type amino acid at position 72), CT-S 109 (where serine is substituted for the wild-type amino acid at position 109), and PT-K9/G129 (where lysine is substituted for the wild-type amino acid at position 9 and glycine substituted at position 129) (see, e.g., International Publication Nos. W093/13202; W092/19265; WO 95/17211; WO 98/18928 and WO 01/22993); and (3) other substances that act as immunostimulating agents to enhance the effectiveness of the composition; oligodeoxy nucleotides containing immunostimulatory CpG motifs (Cpg); or combinations of any of the above.

The invention also provides a pharmaceutical kit comprising one or more containers comprising one or more of the pharmaceutical compositions of the invention. Additional materials may be included in any or all kits of the invention, and such materials may include, but are not limited to buffers, water, enzymes, tubes, control molecules, etc., or any combination thereof The kit may also include instructions for the use of the one or more pharmaceutical compounds or agents of the invention for the prevention or treatment of diseases (for example, diseases associated with immunodeficiency viruses, e.g, AIDS). In some embodiments, the kit comprises one or more syringes, each containing a therapeutic dose of a recombinant herpes virus vector. In some embodiments, the kit comprises one or more vials each vial containing one or more therapeutic doses of a recombinant herpes virus vector, optionally further comprising one or more syringes, wherein a syringe is useful for extracting a therapeutic dose from a vial and delivering the therapeutic dose to a subject. Typically, the kit will contain instructions for administering the recombinant herpes virus vector to a subject. In some embodiments, the kits further comprise compositions (e.g., alcohol wipes) for preparing (e.g., sterilizing) the site of injection on the subject, and/or for sterilizing the vial at the site of syringe penetration. In some embodiments, the kit further comprise a receptacle for disposing of used syringes.

### Viral Vaccine Administration

The compositions disclosed herein can be administered to a subject to produce a prophylactic and/or therapeutic response. In some embodiments, a composition can be used to treat or prevent HIV infection.

As used herein, subject, also referred to as an individual, is any member of the subphylum chordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered. The system described above is intended for use in any of the above vertebrate species, since the immune systems of all of these vertebrates operate similarly. In some embodiments, a subject is a human clinical patient having, or at risk of having, an immunodeficiency virus infection. In some embodiments, a subject is a human clinical patient having, or at risk of having, an HIV infection. However, in some embodiments, a subject is a healthy subject who may be at risk of infection and a composition of the invention is provided as a substitute or supplement to a vaccine.

Compositions will include effective amounts of recombinant viral vector, e.g., amounts sufficient to provide a protective effect and/or to treat, reduce, or prevent an infection (e.g., an immunodeficiency virus infection). An effective amount can be evaluated by comparing the protective effect of a neutralizing agent of the invention to the protective effect of a vaccine (e.g., a control vaccine). In contrast, an immune response to a vaccine can be detected by looking for antibodies to a pathogenic organism (e.g., an immunodeficiency virus antigen) used (e.g., IgG or IgA) in patient samples (e.g., in blood or serum, in mesenteric lymph nodes, in spleen, in gastric mucosa, and/or in feces). The precise effective amount for a given patient will depend upon the patient's age, size, health, the nature and extent of the condition, the precise composition selected for administration, the patient's taxonomic group, the capacity of the patient's immune system to synthesize antibodies, the degree of protection desired, the formulation of the vaccine, the treating physician's assessment of the medical situation, and other relevant factors. Thus, it is not useful to specify an exact effective amount in advance, but the amount will fall in a relatively broad range that can be determined through routine trials, and is within the judgment of the clinician. For purposes of the present invention, an effective dose will typically be above 10⁷, 10⁸, or 10⁹ recombinant viral particles (e.g., colony forming units).

The recombinant gamma herpes viruses of the invention can be administered by any conventional route, including injection or by gradual infusion over time. The administration may, for example, be oral, intravenous, intratumoral, intraperitoneal, intramuscular, intracavity, subcutaneous, or transdermal. In preferred embodiments, the administration is intramuscular or subcutaneous. In some embodiments, administration may be via a nasal spray.

The viruses of the invention may be administered alone, in combination with one or more vaccines, and/or in combination with other immunotherapeutic agents and/or treatments. As used herein in combination includes administration together (e.g., in the same composition) and independently (in separate compositions). Compositions administered in combination may, or may not, be delivered at the same interval. In some embodiments, delivering a vaccine in combination with a immunotherapeutic agent may comprise delivering the vaccine on a first day and delivering the immunotherapeutic agent on a second day. In some embodiments, delivering a vaccine in combination with a immunotherapeutic agent may comprise delivering both the vaccine and the immunotherapeutic agent on the same day (e.g., at approximately the same time of the day). However, these examples are not meant to be limiting and other variations of delivering a vaccine in combination with a immunotherapeutic agent are possible. Similarly, one or more recombinant viruses expressing neutralizing agents may be delivered alone or in combination with other compositions described herein. In some embodiments, the interval between administrations of different prophylactic and/or therapeutic agents may be approximately 1 minute, 30 minutes, 1 hour, 3 hours, 6 hours, 12 hours, 1 day, 1 week, 2 weeks, 1 month, 3 months, 6 months, 1 year, 3 years, 6 years, 10 years, or more. In some embodiments, a vaccine is administrated to a subject before at least one immunotherapeutic agent. In some embodiments, a vaccine is administered to a subject after at least one immunotherapeutic agent is administered to the subject. Similarly, neutralizing agents may be delivered before or after vaccines or other therapeutic agents.

In some embodiments, multiple administrations (e.g., prime-boost type administration) will be advantageously employed. For example, recombinant gamma herpes virus vectors expressing one or more neutralizing agents and/or immunodeficiency virus antigen(s) of interest are administered (e.g., a prime administration). Subsequently, the same and/or different neutralizing agents and/or HIV antigen(s) are administered, for example using a second recombinant gamma herpes virus vector (e.g., a boost, or booster, administration). The former is also referred to herein as priming and the latter is also referred to herein as boosting.

Alternatively, a composition comprising neutralizing agents and/or immunodeficiency virus antigens are administered as either a prime or a boost. Multiple boost administrations (boosters) may also be administered. The appropriate interval between priming and boosting will be apparent to one of ordinary skill in the art. In some embodiments, the interval between priming and boosting, or between consecutive boosters, is approximately 1 day, 1 week, 2 weeks, 1 month, 3 months, 6 months, 1 year, 5 years, 10 years, 15 years, 20 years, or more.

In some embodiments, a prime-boost regime includes two or more administrations of recombinant herpes viral vectors encoding one or more immunodeficiency virus neutralizing agents and/or antigens (e.g., HIV antigens) followed by one or more administrations of immunodeficiency antigens themselves (e.g., a pharmaceutical composition comprising a pharmaceutically acceptable carrier and one or more immunodeficiency virus antigens). For example, one or more administrations of recombinant gamma herpes viral compositions may be followed by one or more administrations of immunodeficiency antigens that are able to stimulate the cellular and humoral aspects of the immune system and elicit immune responses capable of treating or preventing a immunodeficiency virus infection. The concentration of protein in each dose of immunodeficiency antigen may vary from approximately 1 microgram to over 1,000 micrograms (or any value therebetween), preferably between about 10 micrograms and 500 micrograms.

In some embodiments, DNA priming achieves protection against the acute phase of infection. In some embodiments, DNA priming produces anti-Env antibodies. However, the invention is not so limited and DNA priming to produce antibodies to any of the immunodeficiency virus proteins disclosed herein is embraced by the current invention. Methods for DNA priming are known in the art as exemplified in Wang S, J Virol. 2005 June; 79(12): 7933-7937. Other suitable priming methods to enhance immune response will be apparent to one of ordinary skill in the art.

It should be appreciated that priming and boosting using neutralizing agents alone is not a typical priming and boosting since the second administration does not technically boost the first one. Rather, the second administration may be used to add a further dose of recombinant virus (e.g., after a period of months or years) to keep the recombinant virus at levels sufficient for protection or therapy.

### EXAMPLES

The following non-limiting examples relate to recombinant herpes virus based vaccines. Examples 1-6 illustrate how a herpes virus (e.g., a gamma herpes virus) can be used to deliver one or more proteins to a subject. Whereas, example 7 relates more specifically to herpes virus based vaccines that express disease neutralizing agents. In aspects of the invention, one or more recombinant viruses may be engineered using techniques illustrated in the following examples.

### Example 1

### Recombinant Gamma Herpes Virus Construction

Certain examples disclosed herein relate to Rhesus monkey rhadinovirus (RRV) a gamma-2 herpesvirus closely related to HHV-8 (KSHV). RRV Genome length is about 130,733 bp with >84 ORFs and the overall gene organization very similar to KSHV. RRV replicates lytically and to high titers in rhesus fibroblasts, and is a natural infectious agent of rhesus monkeys that persists in B cells. A high prevalence of RRV is detected in rhesus monkeys at NEPRC and at other colonies.

Cell Culture:

Human embryonic kidney cells (293T) were maintained on Dulbecco modified Eagle medium (DMEM) supplemented with 10% fetal calf serum, 2 mM L-glutamine, penicillin-streptomycin (50 IU and 50 μg/ml, respectively) at 37° C. in a humidified incubator with 5% CO2. Rhesus macaque skin fibroblasts (RF) were maintained in DH20 medium (DMEM supplemented with 20% fetal calf serum, 2 mM L-glutamine, penicillin-streptomycin [50 IU and 50 μg/ml, respectively] and 10 mM HEPES) at 37° C. in a humidified incubator with 5% CO2.

Recombinant Virus Construction:

Cosmid libraries were constructed from purified RRV DNA. To facilitate heterologous antigen and control gene (e.g., GFP, SEAP) insertion, the ah28 cosmid (as described in Bilello J P, Journal of Virology, February 2006, p. 1549-1562, Vol. 80, No. 3) was digested with AscI and HindIII to remove excess overlapping genomic RRV sequences. The remaining fragment was digested with Klenow fragment to produce blunt ends and ligated, yielding ah28 A/H. Complementary oligonucleotides, 5′-CTAGTTGTTTAAACGGGGCGCCGGA-3′ (SEQ ID NO: 1) and 5′-CTAGTCCGGCGCCCCGTTTAAACAA-3′ (SEQ ID NO: 2), were annealed at 55° C. and phosphorylated using T4 polynucleotide kinase, forming an adaptomer. The adaptomer featured a cut Spel site at each end flanking a central PmeI site. The ah28 A/H cosmid was linearized with SpeI and dephosphorylated using CIP. Subsequently, the linearized ah28 A/H cosmid was ligated to the SpeI-PmeI-SpeI adaptomer, yielding ah28 A/H-PmeI.

Control Gene Insertion:

To generate the ah28 A/H-CMV-GFP cosmid, ah28 A/H-PmeI was digested with PmeI and dephosphorylated with CIP. The cytomegalovirus (CMV)-GFP cassette was obtained by PCR amplification of pEGFP-C1 (where EGFP is enhanced GFP; BD Biosciences Clontech, Palo Alto, Calif.)'. The amplified product contained the CMV-GFP cassette flanked by PmeI restriction sites at its ends. The PCR fragment was digested with PmeI and ligated to the linearized ah28 A/H-PmeI cosmid, yielding ah28 A/H-CMV-GFP. The pCMV/SEAP (Tropix, Inc., Bedford, Mass.) expression plasmid was modified to contain PmeI restriction sites flanking the CMV-directed transgene. Complementary oligonucleotides, 5′-GATCTAGCTTTGTTTAAACGGGGCGA-3′ (SEQ ID NO: 3) and 5′-GATCTCGCCCCGTTTAAACAAAGCTA-3′ (SEQ ID NO: 4), were annealed at 55° C. and phosphorylated using T4 polynucleotide kinase, forming an adaptomer. The adaptomer featured a cut BglII site at each end flanking a central PmeI site. The pCMV-SEAP plasmid was linearized with BglII and dephosphorylated with CIP. Subsequently, the linearized pCMV-SEAP plasmid was ligated to the BglII-PmeI-BglII adaptomer, yielding pCMV-SEAP BP. A KpnI-PmeI-KpnI adaptomer, from complementary oligonucleotides 5′-CAGCTTTGTTTAAACGGGGCGGTAC-3′ (SEQ ID NO: 5) and 5′-CGCCCCGTTTAAACAAAGCTGGTAC-3′ (SEQ ID NO: 6), was annealed at 55° C. and phosphorylated using T4 polynucleotide kinase. This adaptomer featured a cut KpnI site at each end flanking a central PmeI site. The pCMV-SEAP BP plasmid was linearized with KpnI and dephosphorylated with CIP. Subsequently, the linearized pCMV-SEAP BP plasmid was ligated to the KpnI-PmeI-KpnI adaptomer, yielding pCMV-SEAP PmeIx2. The pCMV-SEAP PmeIx2 plasmid was digested with PmeI and ligated to the linearized ah28 A/H-PmeI cosmid, yielding ah28 A/H-CMV-SEAP.

SIV Antigen Gene Insertion:

Expression cassettes for SIV239-Gag, SIV239-Env, and a tat-rev-nef fusion protein of SIV239 were used for insertion into the RRV genome. In each case, the site of insertion was between the left terminal repeat sequences of RRV and the first RRV open reading frame called R1. The point of insertion is the same as for insertion of the control reporter genes Green Fluorescent Protein (GFP) and secreted alkaline phosphatase (SEAP). Other sites of insertion could be used. Nonessential auxiliary genes could also be displaced by the expression cassettes.

The enhancer/promoters that were used are as follows:
SIV Gag-CMV immediate-early
SIV Env-EIF-1alpha
SW tat-rev-nef-SV40

Other enhancer/promoters could be used, including the virus' own enhancers/promoters.

The poly A 3′end signals that were used are as follows:
SW Gag-HSV TK poly A
SW Env-bovine growth hormone poly A
SW tat-rev-nef-SV40 poly A

The direction of gene orientation relative to the R1 reading frame is as follows
SIV Gag-sense
SW Env-sense
SW tat-rev-nef-antisense

DNA Sequencing:

Cosmid and plasmid constructs were sequenced with a CEQ 8000 Genetic Analysis System using a dye terminator cycle sequencing kit as specified by the manufacturer (Beckman Coulter, Fullerton, Calif.).

Cotransfection and Virus Preparation:

Prior to transfection, the cosmids were digested overnight with the ICeuI homing endonuclease, removing the RRV26-95 sequence from the pSuperCos 1 backbone vector. The cosmid DNA was precipitated by adding 3 volumes of 5% 3 M sodium acetate-95% ethanol and incubating for >1 h at −20° C. The DNA was then pelleted by centrifugation for 10 min at maximum speed in a microcentrifuge. The pellets were washed in 70% ethanol, dried, and rehydrated in distilled water. One day postseeding, 293T cells (4.5×105 cells/well in six-well plates) were transfected with different combinations of digested overlapping cosmids (0.4 μg of each cosmid) using Transfectin reagent (Bio-Rad Laboratories, Hercules, Calif.) following a scaled-down procedure. As a positive control, 0.25 μg of whole viral RRV DNA isolated from column-purified RRV26-95 was transfected in the same manner. At 5 days posttransfection, cell-free culture supernatant was collected and stored at 4° C. To amplify recombinant stocks generated in 293T cells, fresh RF cultures were inoculated with 1 ml of the supernatant collected from the 293T transfections. Inoculated RF cultures were passaged until the emergence of viral plaques was observed in the cultures, and then cultures were maintained without splitting until complete lysis of the RF monolayer. High-titer recombinant RRV stocks were subsequently generated in fresh RF cultures.

Isolation and analysis of RRV DNA. For each RRV virus, supernatant collected following complete lysis of RRV-infected RFs was subjected to low-speed centrifugation to remove cellular debris. The supernatant was then filtered through a 0.45-μm-pore-size filter to remove any additional debris. The filtered supernatant was then centrifuged for 3 h at 45,000×g in a Sorvall type 19 rotor to pellet the virus. The crude virus was resuspended in Tris-EDTA buffer and lysed by adding 0.1 vol. 1% N-lauroylsarcosine and proteinase K and incubating at 60° C. for 1 h. The mixture was extracted twice with phenol-chloroform, followed by four chloroform washes. The DNA was recovered by precipitation with 2.5 volumes of 5% 3 M sodium acetate-95% ethanol, rinsed in 80% ethanol, and resuspended in Tris-EDTA buffer. Viral DNA was digested with restriction endonucleases, separated on a 0.5% agarose electrophoretic gel, and stained with ethidium bromide.

Plaque Assay:

The titers of parental RRV26-95 and recombinant RRV stocks were determined as previously described (DeWire, S. M., et al., Virology 312:122-134). Briefly, cell-free culture supernatant was collected following complete lysis of RRV-infected RFs. Fresh RFs were seeded into 12-well plates at 2×105 cells/well. The following day, 10-fold serial dilutions of the virus-containing supernatant were made in DH20 medium. The medium was removed from the RF cultures and replaced with 200 μl of diluted virus/well. Cultures were then incubated for 1 h at 37° C. with gentle rocking every 15 min. After 1 h, 2 ml of Hank's buffered saline solution (HBSS) was added to each well and subsequently aspirated. Two milliliters of overlay medium (1:1 ratio of 2× DMEM and 1.5% methyl-cellulose [Sigma, St. Louis, Mo.] supplemented with 2% fetal calf serum) was then applied, and the cultures were incubated at 37° C. and 5% CO2 for 1 week. Overlay medium was then aspirated, and a staining solution (0.8% crystal violet in 50% ethanol) was applied for 10 min. Each well was then washed five times with distilled water, and the number of plaques at each dilution of inoculum was determined.

Quantitative Real-Time PCR:

At the indicated time postinfection (p.i.), viral DNA was isolated from 200 μl of cell-free culture supernatant from each sample using the QiaAmp DNA Blood Mini Kit (QIAGEN, Valencia, Calif.) according to the manufacturer's protocol. Tenfold serial dilutions (ranging from 1 to 106 plasmid copies/reaction) of pcDNA3.1/RRV-pol were used in each assay to generate a standard curve for genome copy number. The pcDNA3.1/RRV-pol plasmid was constructed by PCR amplification of RRV polymerase (Pol) from the ah28 cosmid using the primers 5′-CCCAAGCTTATGGATTTCTTTAACCCGTACC-3′ (SEQ ID NO: 7) and 5′-CGCGGATCCTCACGAGAACAGCTTATACGGGAC-3′ (SEQ ID NO: 8). The amplified product contained the RRV Pol gene flanked by an upstream HindIII site and a BamHI site downstream. The resulting PCR product and the pcDNA3.1 plasmid were digested with HindIII and BamHI, gel purified, and ligated together to generate pcDNA3.1/RRV-pol. Quantitative PCRs were performed using the iQ Supermix kit and the MyiQ Single Color Real-Time PCR Detection System (Bio-Rad). The 94-bp amplicon internal to the RRV pol sequence was amplified using the primers 5′-CCGCTTTCTGTGACGATCTG-3′ (SEQ ID NO: 9) and 5′-AGCAGACACTTGAACGTCTT-3′ (SEQ ID NO: 10) and the probe 5′-6FAM-CCAGGATCACTGCGGACCTGTTCC-TAMRA-3′ (SEQ ID NO: 11). Amplification was performed using the following conditions: 95° C. for 3 min, followed by 50 cycles of 95° C. for 30 s and 60° C. for 30 s. Reactions were performed in triplicate and no-template controls were included in the analysis. The number of RRV genome copies/reaction was calculated from the equation for the standard curve using the MyiQ real-time PCR detection system software.

Reporter Gene Expression:

SEAP expression was quantitated using the Phosphalight kit (Applied Biosystems, Foster City, Calif.). GFP expression was observed by fluorescence microscopy, and emission intensity was quantitated using the Victor3V 1420 Multilabel Counter with 480-nm excitation and 510-nm emission filters (PerkinElmer, Wellesley, Mass.).

RRV ELISA:

RRV26-95 was pelleted and column purified as previously described (Desrosiers, R. C., et al., 1997 J. Virol. 71:9764-9769). Purified virus was lysed in 0.1 volume of 10% Triton X-100, and protein concentration was determined using a BCA (bicinchoninic acid) Protein Assay Kit (Pierce Biotechnology, Rockford, Ill.). ELISA plates were coated with 2 μg/ml RRV26-95 lysate for 1 h at room temperature. ELISAs were then performed as previously described (Kodama, T., et al., 1989. AIDS Res. Hum. Retrovir. 5:337-343.).

Viral Neutralization:

RF cells were seeded into 24-well plates at 1×105 cells/well. One day postseeding, RRV-SEAP (0.006 PFU/cell) or RRV-GFP (0.04 PFU/cell) was incubated with various dilutions of heat-inactivated rhesus monkey serum or concentrations of purified rhesus monkey immunoglobulin G (IgG) in a total volume of 200 μl for 3 h at 37° C. with constant gentle rocking. The heat-inactivated serum and purified IgG were diluted in DH20 medium. RRV-SEAP or RRV-GFP was also diluted in DH20 medium without antibody to serve as a no-antibody control for virus neutralization. After the preincubation period, RF cultures were inoculated with medium alone, virus alone, or the virus-serum or virus-IgG mixture. At 16 to 20 h p.i., cultures were rinsed five times with HBSS and refed with DH20 medium. At the indicated day p.i., cultures were examined for either SEAP or GFP expression.

Purification and Depletion of Serum IgG:

Rhesus monkey serum was diluted 1:5 in HBSS and centrifuged at 640×g for 5 min at room temperature to remove debris. For large-scale IgG purification, the clarified serum was decanted into a column containing 300 μl of protein A-Sepharose (Amersham Biosciences, Piscataway, N.J.). The diluted serum was allowed to flow through the column. The column was then washed with 50 volumes of phosphate-buffered saline (PBS), and IgG was eluted with ImmunoPure IgG Elution Buffer (Pierce) into 0.1 volumes of 10×PBS to neutralize the elution buffer. IgG was concentrated using a Viva Spin concentrator (50 kMWCO PES; Vivascience, Hannover, Germany) and dialyzed overnight in PBS at 4° C. The IgG protein concentration was determined using the BCA Protein Assay Kit (Pierce) according to the manufacturer's instructions. Furthermore, the IgG concentration in serum was approximated by batch immunoprecipitation. Based on the IgG concentration determined by BCA analysis of the purified antibody fraction, known amounts of IgG and serum were diluted to a final volume of 200 μl in PBS. Fifty microliters of a protein A and protein G (protein A/G)-Sepharose mixture (1:5) was added, and the samples were mixed overnight at 4° C. Afterwards, the protein A/G-Sepharose was pelleted in a microcentrifuge, and the IgG-depleted supernatant was removed. The protein A/G-Sepharose pellets were rinsed four times with PBS, resuspended in Laemmli buffer, and boiled for 5 min. The Sepharose was pelleted in a microcentrifuge, and the supernatant was electrophoresed through a 12% polyacrylamide-sodium dodecyl sulfate gel. The gel was then stained with Coomassie blue for 30 min and destained in methanol-acetic acid.

### Example 2

### RRV-SIV Mediated Immunization

Rhesus monkey rhadinovirus (RRV) is a close monkey relative of the human Kaposi sarcoma-associated herpesvirus (KSHV). The original isolation and characterization of RRV was first described by the Desrosiers laboratory (J Virol 71: 9764-9769, 1997). RRV and KSHV are members of the gamma-2 (rhadinovirus) subfamily of herpesviruses, quite distinct from members of the alpha (e.g. herpes simplex virus, varicella zoster virus, B virus) and beta (e.g. cytomegalovirus and HHV-6) subfamilies. Herpesviruses in different subfamilies have quite different properties, including the principal cell types in which they establish long-term persistence and the complement of genes that they carry. All herpesviruses have long double-stranded DNA genomes and all persist for the lifetime of the infected host. Persistence of immune responses to antigens expressed by a recombinant herpesvirus may be a desirable feature for the efficacy of a recombinant virus vector vaccine. Moreover, persistent or periodic expression of antigen in the lymphoid compartment may be particularly important for influencing the quality or the strength of the immune response. Gamma-2 herpesviruses could be superior to other alpha or beta herpesviruses for recombinant vaccines with respect to the nature of immune responses to expressed foreign antigens. Gamma-2 herpesviruses persist largely in lymphoid tissues, mostly B cells, which may contribute to this response.

Five rhesus monkeys were recently inoculated with RRV-SIV recombinant. The RRV-SIV expressed SIV Gag, Env, and a tat-rev-nef fusion protein. A different promoter was used for each SIV expression cassette. The site of insertion was the same as what was used in Bilello et al J Virol 80, 1549-1562, 2006 for expression of the reporter genes for green fluorescent protein (GFP) and secreted alkaline phosphatase (SEAP) and is described herein in Example 1. Four of the five immunized monkeys were MamuA*01-positive so that we could conveniently monitor anti-SIV CD8 responses by tetramer staining. Two of these four monkeys were intentionally already positive for RRV by natural infection so that we could monitor the impact of prior RRV status on the “take” of the RRV-SIV and on the kinetics and magnitude of the anti-SIV immune response. The anti-SIV responses at this early stage measured by tetramer staining have been absolutely spectacular. In fact, no other vector approach has been able to achieve the level of anti-SIV responses that we have observed with this single inoculation of RRV-SIV (see summary of results attached). In animal 175-91, 13.2% of all CD8 cells stained specific for the one epitope called CM9 in gag at three weeks post immunization and 3.3% of all CD8 cells stained positive for the MamuA*01-restricted epitope in tat also at three weeks. The two monkeys that were already RRV-positive at the time of immunization also showed anti-SIV responses but they were lower in magnitude and they peaked at two weeks rather than three weeks.

TABLE 4
Detection of SIVgag by IHC in
peripheral lymph node biopsies

| Biopsy | Animal | Vaccine | RRV status | IHC gag |
|---|---|---|---|---|
| B06-346 | 166-91 | RRV-SIV | neg | pos (+) |
| B06-348 | 175-91 | RRV-SIV | neg | pos (++) |
| B06-343 | 128-04 | RRV-SIV | neg | pos (+) |
| B06-349 | 247-04 | RRV-SIV | pos | neg |
| B06-345 | 232-04 | uncloned RRV | neg | neg |
| B06-344 | 309-04 | cloned RRV | neg | neg |
| B06-347 | 440-02 | RRV-SIV | pos | pos (+) |

TABLE 5
Viral Loads

| | Controls | Vaccinated | Fold Reduction |
|---|---|---|---|
| wk 2 mean | 37,000,000 | 1,020,000 | 37 |
| wk 2 range | 27-5⁶ × 10⁶ | 0.017-1.6 × 106 | |
| wk 4 mean | 3,130,000 | 50,000 | 62 |
| wk 4 range | 1.3-6.5 × 10⁶ | 13-67 × 10³ | |

### Example 3

### Design of Vaccine Study

5 monkeys received RRV-SIV
3 of the 5 were RRV-naive
2 of the 5 were already RRV-positive
4 of the 5 were Mamu-A*01-positive
1 additional monkey received cloned, nonrecombinant RRV-26-95
1 additional monkey received uncloned RRV-26-95 stock

### Example 4

### Rhesus Monkey Immunization

One strain of rhesus monkey rhadinovirus (RRV) was genetically engineered to express SW gag protein, another to express SIV env protein and another to express an SW rev-tat-nef fusion protein. Three RRV-negative rhesus monkeys and two RRV+ rhesus monkeys were inoculated with a mixture of these three RRV-SIV recombinants. Cellular responses in the RRV-naïve monkeys to the gag CM9 epitope and the tat SL8 epitope measured by MHC-tetramer staining were spectacular. Furthermore, these responses persisted for the 19 weeks of measurement prior to challenge. Responses in the RRV+ monkeys were diminished but still measurable. The five vaccinated monkeys and three unvaccinated controls were challenged at 18 weeks intravenously with a controlled dose of SIV239. Viral load reductions in the five vaccinated monkeys were 37 fold at week 2 and 62 fold at week 4, both statistically significant.

### Example 5

### KSHV-HIV Mediated Immunization of Humans

A gamma-2 herpesvirus has been demonstrated to be useful as a vector to express lentiviral (AIDS virus) genes. Furthermore, we have found that monkeys immunized with gamma-2 herpesviruses expressing the Gag protein, Envelope protein, and a tat-rev-nef fusion protein of the simian immunodeficiency virus (SIV) made anti-SIV immune responses of very high magnitude, that theses immune responses persisted at surprisingly impressive levels, and we expect that immunized monkeys will be subsequently protected against challenge with pathogenic SIV. These discoveries provide new AIDS vaccine approaches for humans, namely recombinant gamma-2 herpes viruses that are capable of expressing immunogenic proteins of the human immunodeficiency virus (HIV). This discovery statement includes use of derivatives of the human Kaposi sarcoma-associated herpesvirus (KSHV; also called human herpesvirus 8, HHV-8) as a vaccine approach for AIDS. Based on an extensive literature describing superinfection by herpesviruses and our results with RRV-SIV in monkeys already infected with RRV, initial trials for safety and immunogenicity can be performed in subject who are already positive for KSHV with a reasonable expectation for anti-HIV responses. KSHV can be attenuated by any of a number of potential gene deletions using methods well known in the art of attenuated virus generation.

### Example 6

### RRV-SIV ENV Vector Design

RRV-SIVcmv-env/fl
RRV-SIVcmv-env/tr
RRV-SIVcmv-env/sec
RRV-SIVcmv-env/cd-rrv

### Example 7

### Recombinant Gamma Herpes Viruses that Express Disease Neutalizing Agents

Two rhesus monkeys were inoculated with recombinant RRV viruses that contain a transgene that expresses, from a CMV promoter, a single-chain Fv fragment that specifically binds to the Env protein of SW. The site of insertion of the transgene was the same site which was used in Bilello et al J Virol 80, 1549-1562, 2006 for insertion of reporter genes, as described above. The recombinant RRV viruses were produced essentially as described in Example 1 and, prior to inoculation of the monkeys, expression of functional single-chain Fv fragments was validated in vitro by infecting cultures of primary Rhesus monkey fibroblasts (obtained from skin punch biopsies) and EBV immortalized Rhesus monkey B-cells (obtained from ATCC) and testing the cultures for reactivity against the Env protein. For the in vivo studies, after inoculation of the two Rhesus monkeys with the recombinant RRV viruses, serum samples were obtained from each monkey at 2 week intervals up to 16 weeks and specific reactivity against Env protein was detected. These experiments indicated that recombinant virus produced functional Single-Chain Fv fragments in vitro and that the virus can be safely administered to rhesus monkeys to produce functional Single-Chain Fv fragments in vivo.

This invention is not limited in its application to the details of construction and the arrangement of components set forth in the description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items. All of the references cited in this disclosure are incorporated herein by reference.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. A composition comprising a recombinant herpes virus that encodes at least one neutralizing agent, wherein the neutralizing agent interferes with infection or proliferation of a pathogenic organism in a subject.

2. The composition of claim 1, wherein the pathogenic organism is a virus.

3. The composition of claim 2, wherein the virus organism is an immunodeficiency virus.

4. The composition of claim 3, wherein the immunodeficiency virus is selected from HIV-1 and HIV-2.

5. The composition of claim 4, wherein the HIV-1 is a subtype selected from: A, B, C, D, F, H, and O.

6. The composition of claim 4, wherein the HIV-2 is a subtype selected from: A and B.

7. The composition of claim 1, wherein the herpes virus is a gamma herpes virus.

8. The composition of claim 7, wherein the gamma herpes virus is HHV-8.

9. The composition of claim 1, wherein the neutralizing agent is a nucleic acid or protein that interferes with infection of the subject by the pathogenic organism.

10. The composition of claim 9, wherein the neutralizing agent inhibits CCR5 and/or CD4.

11. The composition of claim 9, wherein the neutralizing agent is a decoy molecule that inhibits viral entry into a host cell in the subject.

12. The composition of claim 9, wherein the neutralizing agent is an antibody.

13. The composition of claim 12, wherein the antibody is a broadly neutralizing antibody that binds to one or more HIV antigens.

14. The composition of claim 13, wherein the one or more HIV antigens are Gag, Pol, Env, Tat, Rev, Vif, Vpr, Vpu, Nef, or Vpx antigens.

15. The composition of claim 1, wherein the neutralizing agent is encoded by a nucleic acid that is operably joined to a promoter selected from: an SV40 early promoter, an SV40 late promoter, a CMV immediate early promoter, an EF1 promoter, and a promoter of an endogenous gamma herpesvirus gene.

16. The method of claim 1, wherein the subject is human.

17. A method of protecting a subject from infection by a pathogenic organism, the method comprising administering to a subject at risk of infection by a pathogenic organism an effective amount of a recombinant herpes virus that encodes at least one neutralizing agent, wherein the neutralizing agent interferes with infection of the subject by the pathogenic organism.

18. A method of reducing the viral load of an immunodeficiency virus in a subject, the method comprising administering to a subject infected with an immunodeficiency virus, an effective amount of a recombinant herpes virus that encodes at least one neutralizing agent, wherein the neutralizing agent interferes with proliferation of the immunodeficiency virus in the subject.
